(19) United States
(12) Patent Application Publication
(10) Pub. No.: US 2024/0189234 A1
(43) Pub. Date: Jun. 13, 2024

(54) SILICON PARTICLES FOR DRUG DELIVERY

(71) Applicant: NACAMED AS, Askim (NO)

(72) Inventors: Werner FILTVEDT, Tomter (NO); Jo KLAVENESS, Oslo (NO); Hennie Marie JOHNSEN, Oslo (NO)

(21) Appl. No.: 18/556,513

(22) PCT Filed: Apr. 22, 2022
(86) PCT No.: PCT/EP2022/060773

§ 371 Date: Oct. 20, 2023

(30) Foreign Application Priority Data
Apr. 23, 2021 (GB) 2105833.4

(51) Int. Cl. A61K 9/16; A61K 9/20; A61K 9/50; A61K 31/155; A61K 31/165; A61K 31/343; A61K 31/40; A61K 31/4166; A61K 31/4178; A61K 31/43; A61K 31/436; A61K 31/522; A61K 31/573; A61K 31/635; A61K 31/65; A61K 31/7048; A61K 31/7068; A61K 33/00; A61K 38/13; A61K 47/61

(52) U.S. Cl. CPC A61K 9/1611; A61K 9/1694; A61K 9/2054; A61K 9/2077; A61K 9/5015; A61K 31/155; A61K 31/165; A61K 31/343; A61K 31/40; A61K 31/4166; A61K 31/4178; A61K 31/43; A61K 31/436; A61K 31/522; A61K 31/573; A61K 31/635; A61K 31/65; A61K 31/7048; A61K 31/7068; A61K 33/00; A61K 38/13; A61K 47/61

(57) ABSTRACT

The invention provides a process for preparing silicon particles comprising at least one drug substance, wherein said process comprises the steps: a) preparing silicon particles via chemical vapor deposition (CVD): b) loading the silicon particles prepared in step a) with at least one drug substance.

### FIELD OF THE INVENTION

The present invention relates to a process for preparing silicon particles containing at least one drug substance. In particular, the invention relates to a process wherein the silicon particles are prepared by a process comprising a chemical vapor deposition step. The invention further relates to particles produced by said process, pharmaceutical compositions comprising said particles and to the use of said particles and compositions in therapy, particularly drug delivery.

### BACKGROUND

A drug sold in a pharmacy or used by a patient is generally referred to as a drug product in regulatory and scientific terms. A drug product comprises two groups of components; drug substance(s) and drug additives or excipients. The drug substance is the biologically/pharmacologically active component while the drug additives or excipients are per se inactive components present in the drug product to secure safe, efficient and/or user friendliness of the drug substance. For example, an antibiotic drug substance cannot be injected into the blood stream as a solid material and is therefore typically dissolved in water, as the drug additive or excipient, and prepared as a drug product for injection. The efficacy and safety of a drug product is always dependent on both the nature of the drug substance and the nature of the excipients. This technological field is often referred to as drug formulation or galenic pharmacy.

A drug product can comprise more than one drug substance. Such drug substance(s) may be in the form of a free drug substance, a pharmaceutically acceptable salt, crystalline or amorphous material or can be encapsulated in one or more complex compounds. For a given substance, a range of pharmaceutically acceptable salts can be prepared using different acids or bases in different ratios, resulting in different salts with varying chemical properties such as aqueous solubility, dissolution rate and stability. The therapeutic indications for different salts of a given substance are, however, identical. The same is also true for all possible crystalline forms, amorphous materials and encapsulation complexes for a given drug substance. It is not uncommon for one drug substance to be present in different forms in different drug products such that they can be tailored for different routes of administration.

The field of drug formulation is, for all routes of administration, a complex field of science. For oral administration routes it might in some cases be important that the drug substance is released very fast, for example sublingual tablets comprising nitroglycerin to treat angina in patients with coronary artery disease. The advantages of using sublingual tablets include reduced first pass metabolism and fast action of the drug substance. Oral products releasing a drug substance in the mouth, typically in the form of lozenges, are for example useful for treatment of infections and/or pain in the mouth or throat. Classical capsules and tablets release the drug substance in the gut followed by absorption of the drug substance from the small intestine and in some cases partly from the gut. The main problems associated with this type of pharmaceutical formulation include instability of the drug substance in the gut due to low pH and/or gastric enzymes, local toxicity or irritation of the gut wall caused by the drug substance or very low aqueous solubility/low dissolution rate of drug substance. The first two aspects might be improved by the presence of an enteric coating, ensuring that the drug substance does not dissolve in the stomach but in the intestine. The topic of low aqueous solubility/low dissolution rate is generally a challenge for several older and numerous new potential drug substances. The result is low oral bioavailability, leading to the development of many drug candidates with interesting clinical profiles being abandoned.

For both oral administration and alternative routes, such as parenteral administration, several options using various excipients and mixtures of such excipients have been suggested to secure a safe and effective drug product with good user friendliness. There are currently very many excipients, including various qualities of excipients, used in commercial pharmaceutical products. However, for many drug substances no clinically useful formulations have been developed to date.

One excipient which can be used in pharmaceutical products is silica (silicon dioxide). In the form of hydrophobic silicon dioxide, this compound has several functions, including as an anticaking agent, emulsion stabilizing agent, glidant, suspending agent and viscosity-increasing agent. In the form of anhydrous silicon dioxide this compound may act as an adsorbent, anticaking agent, desiccant, emulsion stabilizing agent; glidant, suspending agent, tablet and capsule disintegrant or viscosity-increasing agent. In the form of hydrated silicon dioxide this compound has several functions including, an adsorbent, anti-adherent, anticaking agent, desiccant, direct compression excipient, flavor enhancer, gelling agent, glidant, plasticizing agent, solubilizing agent, suspending agent, tablet and capsule disintegrant and taste-masking agent.

Silicon comprising particles have also been investigated as potential drug delivery excipients. Typically, these particles are silicon dioxide particles, however, some studies have focused on silicon particles where the silicon is in the form of pure silicon with oxidation number zero (i.e. silicon zero particles) and thus not in the form of silicon oxide. Such particles might, however, comprise some silicon oxide due to native oxidation of the silicon zero surface. The particles are typically prepared by ball milling of silicon materials produced for the electronic industry, such as semiconductors. Most of these silicon particles are porous particles produced by etching of the particles before or after milling, for example by using hydrofluoric acid (HF).

Silicon zero materials can also be prepared by a so-called chemical vapor deposition (CVD) process wherein a silicon comprising gas, for example silane (SiH4), is thermally decomposed to silicon zero solid material. One option is to mill this solid material down to small sized particles. Silicon zero particles produced by centrifuge chemical vapor deposition, are described in, for example WO 2013/048258 and by Lumen et al in Eur. J. Pharm. and Biopharm. 158 (2021)254-265. Lumen et al relates to a study into intravenous administration of silicon particles without any therapeutic drug. The findings demonstrated that the cCVD production method provided nanoparticles with comparable physical and biological quality to the conventional milling method, however the CVD particles were found to be more toxic than non-CVD particles.

The present inventors have unexpectedly found that silicon particles produced by a direct CVD process, such as that described in WO 2013/048258, without a subsequent milling process, are attractive for use in drug delivery. The potential advantages of using such particles in drug delivery compared to state of the art technology within the pharmaceutical field include: high drug loading, improved drug release profile, improved stability and/or good safety profile.

During growth of CVD silicon particles there will be scavenging of both gaseous species and other nuclei. These other nuclei will have grown to nanospheres that upon scavenging will preserve some internal order. If the growth is performed at high temperature >650° C. the particles will become crystalline. If the growth is performed at lower temperature the particles will become amorphous. The amorphous particles may be crystallized after growth, but then even higher temperature will be needed to post-crystallize the particles. The exact post-production crystallization temperature will be dependent on the growth conditions and size of the grown particles. But all pure silicon particles will crystallize above 770° C.

Since all particles are produced from decomposing an electronics grade silane gas of purity 99.999999% SiH₄ purity, the produced material is pure silicon. Since all scavenging and growth is performed in an environment where only Si and H atoms are present, the internal borderlines between domains are pure. This is the case both if the domains are amorphous or crystalline. By investigation by for instance Transmission Electron Microscopy it is possible to see these domains. The domains are especially clear if the sample is crystalline either grown crystalline or post-growth crystallized. The purity, lack of internal oxidation and spherical shape of the primary particles are all inherent properties of particles grown by CVD. The primary difference between c-CVD and other CVD particles is a more narrow size distribution especially in combination with an amorphous structure. It is possible to achieve a narrow size distribution by use of a high energy supply and short growth time for instance by laser or plasma torch growth zone. However, by doing the growth control in this way one will always get a crystalline structure of substantially larger crystals.

The main differences between CVD particles and crushed particles is the spherical nature of the primary particles and lack of sharp edges for the CVD particles. The CVD particles are grown from gas in a process for the sake of clarity may be viewed as the growth of hail. The spherical nature of hail is a result of the same primary growth mechanisms, scavenging of gas and smaller solid-domains that in the end will form the complete hail-sphere. The crushed silicon-particles may be viewed for the sake of clarity as the equivalent of crushing down ice-cubes. Both CVD and crushed particles may include crystalline domains, but for the CVD particles these domains will all be small, of a narrow size distribution, the particles will be spherical and the internal surfaces will be unoxidized and uncontaminated. For crushed particles there may be internal crystalline domains, but of varying size and distribution. The crushed particles are formed by breaking a larger particle and will therefore inherently always have sharp edges. The internal surfaces if any will have seen other atoms than Si and H and will therefore always be more contaminated than direct electronics grade Si particles. The crushing is also challenging to perform without substantial internal oxidation. The easiest analysis method to distinguish between CVD and crushed particles will be Scanning Electron microscopy or Transmission Electron microscopy. Alternatively by X-ray diffraction to identify a fully amorphous structure.

To support the analysis it is possibly to perform a purity measurement by Inductively coupled plasma mass spectrometry (ICPMS) to verify the purity of the particles. Spherical, pure, unoxidized amorphous or nanocrystalline particles will need to be produced by CVD and will not be possible to achieve by crushing.

### SUMMARY OF THE INVENTION

In a first aspect, the invention provides a process for preparing silicon particles comprising at least one drug substance, wherein said process comprises the steps:
a) preparing silicon particles via chemical vapor deposition (CVD);
b) loading the silicon particles prepared in step a) with at least one drug substance.

In a further aspect, the invention provides silicon particles comprising at least one drug substance prepared according to a process as hereinbefore defined.

In another aspect, the invention provides a pharmaceutical composition comprising silicon particles as hereinbefore defined and one or more pharmaceutically acceptable carriers, diluents or excipients.

In a further aspect, the invention provides silicon particles as hereinbefore defined or a pharmaceutical composition as hereinbefore defined for use in therapy.

In yet another aspect, the invention provides silicon particles as hereinbefore defined or a pharmaceutical composition as hereinbefore defined for use in drug delivery.

### Definitions

The term “drug substance” as used herein refers to any biologically and/or pharmacologically active compound including prodrugs thereof. Any stereoisomer, or pharmaceutically acceptable salt or solvate thereof are included in the present term. The term drug substance includes any drug substance with regulatory approval, drug substances in current development and drug substances that have been on the market.

The term “drug product” refers to a composition comprising at least one drug substance and at least one excipient (i.e. a pharmaceutical composition).

The term “regulatory approved” refers to drug products which are or have been regulatory approved for marketing in at least one country.

The term “under regulatory development” refers to drug products that are known to be in development with the aim to be regulatory approved.

The term “pharmaceutical composition” includes “drug product” and refers to a composition comprising at least one drug substance and at least one excipient.

The term “pharmaceutically acceptable” refers to chemical compounds and mixtures thereof that are acceptable to be used in drug products. All excipients used in regulatory approved drug products are pharmaceutically acceptable.

The term “excipient” refers to chemical compounds for use in drug products where said excipients per se are not biologically active in the amount present when the drug product is used according to the intention or regulatory approval.

The term “complex” refers to a compound comprising at least two different molecules that are associated to each other by additional bonds than covalent bonds and classical ionic bonds in simple salts. One typical example is cyclodextrin complexes.

The term “cyclodextrin” refers to compounds of cyclic oligosaccharides, consisting of a macrocyclic ring of glucose subunits joined by α-1,4 glycosidic bonds. α (alpha)-Cyclodextrin comprises of 6 glucose subunits, β (beta)-cyclodextrin comprises of 7 glucose subunits and γ (gamma)-cyclodextrin comprised of 8 glucose subunits. Unsubstituted cyclodextrin (alpha, beta and gamma) compounds are produced from starch by enzymatic process. Substituted cyclodextrin derivatives are produced by a semisynthetic process.

The term “silicon zero comprising particles” refers to particles wherein at least 50% of the silicon is with oxidation level zero and not four as in silica.

The term “low molecular weight compound” refers to compounds with molecular weight below 3000 Dalton.

The term “biological drug substance” refers to drug substances produced by a living organism. The term does not include substances naturally produced by plants. The term includes semisynthetic drug substances like for example drug/toxin conjugates of monoclonal antibodies. The term is a regulatory term.

The term “food additive” refers to food products in any market.

The “silicon particles of the invention” will be understood to be silicon particles comprising at least one drug substance.

The term cCVD-SP is used to denote “centrifuge Chemical Vapor Deposition Silicon Particles” and refers to silicon particles which have been prepared a centrifuge method. In particular, this term refers to silicon particles which have been prepared by a CVD method in a reactor wherein the reactor comprise a reactor body and a rotation device operatively arranged to the reactor, wherein the rotation device is configured to rotate the reactor around an axis during production of said silicon comprising particles.

The term PcCVD-SP is used to denote “porous centrifuge Chemical Vapor Deposition Silicon Particles” and refers to silicon particles which have been prepared by a centrifuge method, followed by a process, such as etching, to prepare the porosity of the particles.

### DETAILED DESCRIPTION

The present invention relates to a process for preparing silicon particles comprising at least one drug substance, wherein said process comprises the steps:
a) preparing silicon particles via chemical vapor deposition (CVD);
b) loading the silicon particles prepared in step a) with at least one drug substance.
   Step a)—Chemical Vapor Deposition (CVD)

A CVD process is a process wherein a gas is converted to a solid material, typically a film, under various conditions. Step a) of the process of the invention preferably involves preparing silicon particles via CVD from a silicon containing reaction gas, such as silane or trichlorosilane.

In a preferred embodiment of the invention, the CVD process in step a) does not comprises a milling step

In particular, the CVD process of step a) is preferably carried out in a reactor wherein the reactor comprises a reactor body and a rotation device operatively arranged to the reactor, wherein the rotation device is configured to rotate the reactor around an axis during production of said silicon comprising particles; hereafter referred as cCVD-SP (centrifuge Chemical Vapor Deposition Silicon Particles).

In a further preferred aspect of the present invention, the CVD process of step a) is carried out in a reactor wherein the reactor comprises a reactor body and a rotation device operatively arranged to the reactor, wherein the rotation device is configured to rotate the reactor around an axis during production of said silicon comprising particles; hereafter referred as cCVD-SP, optionally followed by an etching process to prepare the porosity of the particles. Such particles are here referred to as PcCVD-SP (Porous centrifuge Chemical Vapor Deposition Silicon Particles).

One preferred aspect of the present invention relates to porous non-etched cCVD-SP. Such particles are typically formed by forming stable aggregates of smaller particles.

Another preferred aspect of the invention relates to non-porous non-etched cCVD-SP particles.

Still another preferred aspect of the present invention relaters to porous amorphous non-etched cCVD-SP particles.

Still another preferred aspect of the present invention relaters to non-porous amorphous non-etched cCVD-SP particles.

The etching process for the production of PcCVD-SP from cCVD-SP is similar to other well-known etching processes of silicon particles described in the literature; for example a hydrofluoric acid based method. The particle surface may be modified to exhibit desired characteristics; including chemical or thermal oxidation or coating.

A particularly preferred method for the preparation of the silicon particles in step a) is disclosed in WO 2013048258 and WO2018052318 and is briefly described below.

In this preferred process, step a) is carried out in a reactor comprising a reactor body that can rotate around an axis with the help of a rotation device operatively arranged to the reactor, at least one sidewall that surrounds the reactor body, at least one inlet for reaction gas, at least one outlet for residual gas and at least one heat appliance operatively arranged to the reactor, characterised in that during operation for the manufacture of silicon particles by CVD, the reactor comprises a layer of particles on the inside of, at least, one side wall.

Thus, the process of step a) is preferably characterised by:
producing a particle layer from the silicon containing reaction gas in the reactor or importing particles for the formation of an inner particle layer on the inner wall surface of the reactor,
importing reaction gas for chemical vapour deposition,
nucleating particles inside the reactor chamber

The formed particles scavenges precursor gas and grow until the weight of the particles move them to the wall where the temperature is lower and further growth is suppressed. The particles are then removed from the wall in a gas stream and collected at a filter. The particles are then removed from the filter by sending a gas pressure pulse the other way back wards through the filter. The particles are collected inert for further processing.

Depending on the application the particles may be coated inert or exposed to air to form a thin native oxide layer on the particles. Further processing may include etching of the particles in HF with or without subsequent coating depending on the application. However, preferably, the particles are not subject to an etching process.

In a particle formed from milling of electronic grade silicon wafers the average crystal size of the material will be many orders of magnitude larger than the particle size. For CVD formed particles the average crystal size is tuneable. It is possible to have one or few crystallites within each particle, to have a number of nano-crystallites within each particle or to have a completely un-ordered amorphous structure. This is tuneable by the process and it is therefore both possible to choose a particular crystallinity or average crystallite size for the specific application or according to further processing. For instance will the etching speed depend on the crystallite size and orientation as well as the defect distribution and frequency within each crystal.

The particle degradation time will to some degree depend on the number of crystal interfaces reaching the surface in other words how many oxidation channels the oxidation may propagate along down into the material as well as how imperfect the individual crystals are. The more imperfections and interfaces the easier it is both to reach the individual silicon atoms and to oxidize them. Since these are tuneable properties in a CVD produced material it is thus possible to tune the material to any specific application in a completely different way than for a crushed large crystals material where these properties are given. Especially for applications where rapid bio-degradation is desirable the CVD particles will have a substantial advantage over the classical crushed crystalline silicon.

Step b)—Loading

The silicon particles produced in step a) are subsequently loaded with at least one drug substance in step b). This loading step b) may take place by any suitable method known in the art. Typically, however, step b) involves mixing the silicon particles obtained in step a) with the at least one drug substance(s) in a solvent.

The solvent is ideally one in which the silicon particles are dispersed and the drug substance is, at least partly, soluble. Typically, the solvent is an aqueous solvent (i.e. comprising, preferably consisting of, water).

The mixing step may take place at ambient temperature (e.g. 20 to 30° C.), or at elevated temperature (e.g. 40 to 80° C., such as 50 to 70° C.). Typically, mixing occurs in a sonicator, although it may take place by any suitable method known in the art.

The cCVD-SP and PcCVD-SP comprising at least one drug substance are typically isolated as a dry product by evaporation, freeze drying, fluid bed, spray drying or any other method well known in the art. If the cCVD-SP and PcCVD-SP comprising at least one drug substance are intended for use as non-dried material, the drying step described above is optional.

### Silicon

The silicon in the silicon particles of the present invention (preferably the cCVD-SP and/or PcCVD-SP) is present in at least 50 wt % as elemental silicon (silicon with oxidation number 0), relative to the total weight of silicon. More preferred form of silicon in the silicon particles is at least 70 wt % as elemental silicon, even more preferred at least 80 wt % as elemental silicon, relative to the total weight of silicon. Another preferred aspect related to the form of silicon in the particles is that the amount of elemental silicon and silicon dioxide is more than 80%, more preferably more than 90% most preferably more than 95%, relative to the total weight of silicon.

Silane and other silicon comprising gases used for preparation of the present particles in the CVD process are very toxic. As a component in drugs it is very important that the amount of silicon comprising gas is very low in the present particles. Still another preferred aspect related to the form of silicon in the present particles is therefore that the amount of silicon comprising gas in the particles is less than 10 wt %, more preferably less than 5 wt %, most preferably less than 2 wt % of the total weight of silicon in the particles.

The elemental silicon in the particles of the invention may be in amorphous or crystalline form. The elemental silicon in particles produced by the CVD process is mainly in the form of amorphous elemental silicon at ambient temperature, however, particles comprising crystalline silicon can directly be prepared by CVD at high temperature (e.g. 630° C. and above) and longer reaction times. The particles comprising crystalline silicon prepared from a CVD method typically are in the form of polycrystalline material (crystal size around 1.5 nm) while crystalline milled particles typically consists of one crystal of silicon.

The crystalline versus amorphous form of silicon can routinely be determined by X-ray diffraction analysis (XRD analysis). The amorphous form of silicon can be transformed to crystalline form of silicon by heating to relative high temperatures (e.g. above 650° C.).

In certain embodiments, the elemental silicon is present in a crystalline form, in some embodiments typically more than 60 wt % in the crystalline form and in some embodiments more than 80 wt % in a crystalline form and finally in some embodiments more than 90 wt % in a crystalline form, relative to the total weight of elemental silicon.

In other embodiments, the silicon particles comprise elemental silicon in amorphous form, in some embodiments more than 80 wt %, in some embodiments more than 90 wt %, in some embodiments more than 95 wt % and finally in some embodiments more than 99 wt % in amorphous form, relative to the total weight of elemental silicon.

One preferred embodiment of this aspect of the invention is wherein the silicon particles are cCVD-SP or PcCVD-SP.

One of the most preferred embodiment of this aspect of the invention is wherein the silicon particles are cCVD-SP comprising silicon in amorphous form, such as in the wt % ranges defined above.

Another of the most preferred embodiment of this aspect of the invention is wherein the silicon particles are cCVD-SP that are not produced by an etching process; especially not by an hydrofluoronic (HF) etching process, i.e. the silicon particles are non-etched.

The ultimate form of the most preferred embodiment of this aspect of the invention is wherein the silicon particles comprise amorphous silicon, such as in the wt % ranges defined above, and are non-etched.

### Particle Size

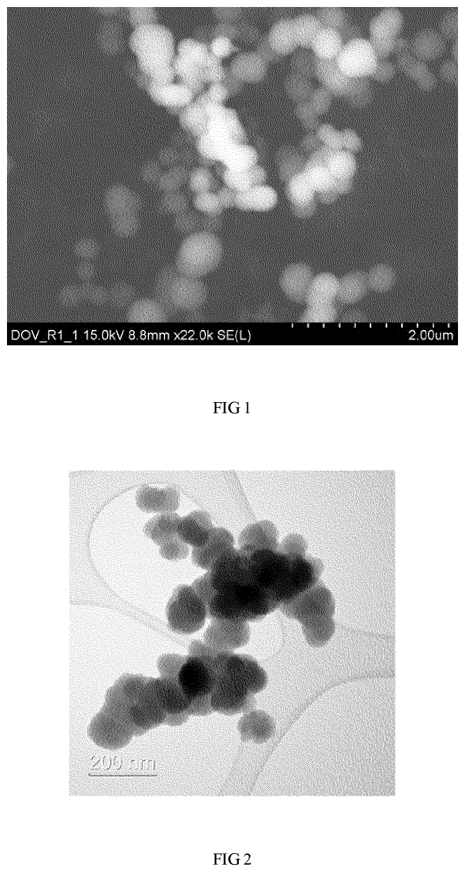

The processes of the invention allow for silicon particles with “tailor made” particle size to be prepared. Typical median diameter average particle sizes for the silicon particles of the invention may be less than 500 nm, such as 30 to 300 nm, using the technique of Dynamic Light Scattering (DLS), for example using instruments like Zetasizer. The given particle sizes are related to the final silicon particles loaded with one or more drug substances and optionally excipients and coating. The particles consist of single particles that are clustered together partly through bonding and partly loosely. FIGS. 1 to 3 show examples of the particles.

The polydispersity index can also vary from almost monodisperse particles to particles with very broad particle size distribution.

The preferred particle size of the silicon particles of the invention will generally vary depending upon indication and route of administration. Particles for intravenous administration should typically have an average particle size of less than 500 nm, more preferably less than 200 nm; for intramuscular injection the average particle size should preferably be less than 10 μm, typically less than 5 μm; for subcutaneous administration and ocular use the average particle size should typically be less than 5 μm; for nasal application the average particle size should typically be less than 50 μm; for intrapulmonary administration (inhalation) the average particle size should typically be less than 15 μm and for oral administration the average particle size should be less than 500 μm.

### Porosity

The silicon particles of the invention can be non-porous (cCVD-SP) or porous (PcCVD-SP). The most preferred particles according to the present invention are porous particles. In all embodiments, it is preferred if the particles are prepared by a non-etching process. Porous particles for drug delivery can be prepared by forming stable aggregates of smaller particles; so-called stable particle clusters.

The porosity of the PcCVD-SP can vary over a large range depending upon choice of drug substance, indication and administration route. The porosity is a measure on the volume of the pores. A PcCVD-SP with porosity of 50% has a porosity volume that is 50% of the total PcCVD-SP volume. The porosity of PcCVD-SP may typically be from 20% to 90%. In certain embodiments, the porosity is more than 40%, typically more than 50%, preferably more than 60%, more preferably more than 70%, especially more than 80% such as 90%. In other embodiments the porosity is preferably around 50% or lower.

The pore size of PcCVD-SP can vary from microporous particles through mesoporous particles to macroporous particles depending on nature of the drug substance, dose of the drug substance, indication, form of the drug product and route of administration. Typical average pore size of PcCVD-SP for loading of drug substances is from 1 nm to 200 nm. In one embodiment of the present invention, the average pore size is 1-10 nm, in another embodiment the typical pore size is 5-20 nm, in still another embodiment, the typical pore size is 10-50 nm and finally, in still another embodiment, the typical pore size is 2-50 nm.

In one embodiment, the particles are microporous. In this embodiment, preferably at least 2 vol % of the pores are micropores, more preferably at least 5 vol %, even more preferably at least 10 vol %, especially at least 20 vol %, such as at least 50 vol %, relative to the total pore volume.

In another embodiment, the particles are mesoporous. In this embodiment, preferably at least 2 vol % of the pores are mesopores, more preferably at least 5 vol %, even more preferably at least 10 vol %, especially at least 20 vol %, such as at least 50 vol %, relative to the total pore volume.

In a further embodiment, the particles are macroporous. In this embodiment, preferably at least 2 vol % of the pores are macropores, more preferably at least 5 vol %, even more preferably at least 10 vol %, especially at least 20 vol %, such as at least 50 vol %, relative to the total pore volume.

### Particle Surface and Coating

The particle surface can typically be in the form of elemental silicon or more preferably in the form of a layer of silicon oxide where the elemental silicon on the particle surface has undergone a natural or a chemical oxidation process. The surface might also be covered by a layer of drug molecules that are covalently or non-covalently bound to the silicon-comprising material. The surface might also be covered by a coating material comprising carbon, preferably in the form of an organic coating. The organic coating might be bonded to the silicon comprising material by covalent or non-covalent bonds. The chemistry of coating of silicon particles is well known in the art.

An optional coating might have one or more different functions, such as:
The coating might protect the silicon particle against degradation
The coating might control the release profile of the drug substance
The coating might affect the in vivo biodistribution of the particles after administration.
The coating might improve the loading of drug substances into silicon comprising particles.
The coating might form basis for covalent attachment of drug substances to the coating material

The coating might from a chemical perspective have one or more of the following properties:
Hydrophilic coating for example in the form of covalently attached polyethylene glycol chains.
Positively charges particle surface at physiological pH. This can typically be obtained by attachment of aliphatic amino groups to the particle surface.
Negatively charges particle surface at physiological pH. This can typically be obtained by attachment of carboxylic groups to the particle surface.
Enzymatically degradable coating. Typical coatings include for example coatings comprising ester groups.
Coatings comprising a monolayer of coating molecules.
Coatings comprising multilayer of coating molecules.
Coatings based on monomer compounds
Coatings based on polymer compounds
Coatings based on phospholipids and/or other lipid derivatives.
Coatings based on proteins, peptides or amino acids or derivatives thereof.
Coatings based on sugar molecules; including, monosaccharides, disaccharides, oligosaccharides including cyclodextrins and polysaccharides.

The surface area of the silicon particles of the invention will vary. The surface area will be much higher for porous particles (PcCVD-SP) than non-porous particles (cCVD-SP). The surface area of the particles prior to loading of the at least one drug substance may be up to 1000 m² per gram particles.

Preferred coatings include surfactants like for example ceteareth, cetearyl, ceteth, cocamide, isosteareth, laureth, lecithin, oleth PEG-20 almond glycerides, PEG-20 methyl glucose sesquistearate, PEG-25 hydrogenated castor oil, PEG-40 sorbitan peroleate, PEG-60 Almond Glycerides, PEG-7 olivate, PEG-7 Glyceryl cocoate, PEG-8 dioleate, PEG-8 laurate, PEG-8 oleate, PEG-80 sorbitan laurate, Polysorbates and Pluronics

### Drug Substance

The silicon particles of the invention comprise one or more drug substances. Whilst the silicon particles may comprise only one drug substance, it is also possible for more than one drug substance to be present, such as two or three drug substances.

A preferred embodiment of the present invention relates to cCVD-SP comprising one drug substance.

A more preferred embodiment of the present invention relates to PcCVD-SP comprising one drug substance.

Another preferred embodiment of the present invention relates to cCVD-SP comprising two drug substances.

A more preferred embodiment of the present invention relates to PcCVD-SP comprising two drug substances.

Another preferred embodiment of the present invention relates to cCVD-SP comprising three or more drug substances.

A more preferred embodiment of the present invention relates to PcCVD-SP comprising three or more drug substances.

The drug substances to be used according to the present invention include any drug substance, regulatory approved drug substance and any drug substance in development for prophylactic use and/or treatment of disease.

In one embodiment, the drug substance if preferably selected from the group consisting of anticancer drugs, drugs with effect on the immune system, antifungal drugs, antibiotics, antiviral drugs, drugs for treatment of CNS related diseases, antidiabetic drugs, drugs for treatment of pain and steroid-based drugs.

One preferred aspect of the present invention relates to drugs for prophylactic use and/or treatment of disease related to the gastrointestinal system and metabolism. Such drug substances are typically included in ATC group A. Drug substances for treatment of diseases related to the gastrointestinal system and metabolism, including anti-infectives and antiseptics for local oral treatment, corticosteroids for local oral treatment and other agents for local oral treatment.

Drug substances for treatment of acid related disorders including antacids, including drugs for peptic ulcer and gastroesophageal reflux disease (GORD) like H2-receptor antagonists, for example cimetidine, ranitidine, famotidine, nizatidine, niperotidine, roxatidine, ranitidine bismuth citrate and lafutidine, including prostaglandins for example misoprostol and enprostil, including proton pump inhibitors for example omeprazole, pantoprazole, lansoprazole, rabeprazole, esomeprazole, dexlansoprazole, dexrabeprazole andvonoprazan, including combinations for eradication of Helicobacter pylori and other drugs for peptic ulcer and gastro-oesophageal reflux disease (GORD) and including other drugs for acid related disorders for example carbenoxolone, sucralfate, pirenzepine, methiosulfonium chloride, bismuth subcitrate, proglumide, gefarnate, sulglicotide, acetoxolone, zolimidine, troxipide, bismuth subnitrate, alginic acid, rebamipide, carbenoxolone and gefarnate.

Drug substances for treatment of functional gastrointestinal disorders including antispasmodics like belladonna alkaloids and derivatives thereof.

Other relevant drug substances include antiemetics like ondansetron and other serotonin (5HT3) antagonists, drug substances for treatment of disorders related to bile and liver, anticonstipation drug substances including laxatives, drug substances for treatment of diarrhea, anti-obesity drug substances and gastrointestinal digestives including enzymes.

Drugs for treatment of diabetes including insulins and analogues including insulins and analogues for injection, fast-acting like for example insulin (human), insulin (beef), insulin (pork), insulin lispro, insulin aspart and insulin glulisine, including insulins and analogues for injection, intermediate-acting like for example insulin (human), insulin (beef), insulin (pork), insulin lispro, including insulins and analogues for injection, intermediate- or long-acting combined with fast-acting like for example insulin (human), insulin (beef), insulin (pork), insulin lispro, insulin aspart, insulin degludec and insulin aspart, including insulins and analogues for injection, long-acting like for example insulin (human) like for example insulin (beef), insulin (pork), insulin glargine, insulin detemir, insulin degludec, insulin glargine and lixisenatide and insulin degludec and liraglutide. Other non-insulin blood glucose lowering drugs including biguanides like for example phenformin, metformin and buformin, sulfonylureas like for example glibenclamide, chlorpropamide, tolbutamide, glibornuride, tolazamide, carbutamide, glipizide, gliquidone, gliclazide, metahexamide, glisoxepide, glimepiride and acetohexamide, including heterocyclic sulfonamides like for example glymidine, including alpha glucosidase inhibitors like for example acarbose, miglitol and voglibose, including thiazolidinediones like for example troglitazone, rosiglitazone, pioglitazone andlobeglitazone including dipeptidyl peptidase 4 (DPP-4) inhibitors like for example sitagliptin, vildagliptin, saxagliptin, alogliptin, linagliptin, gemigliptin, evogliptin and teneligliptin, including glucagon-like peptide-1 (GLP-1) analogues like for example exenatide, liraglutide, lixisenatide, albiglutide, dulaglutide, semaglutide, beinaglutide, including sodium-glucose co-transporter 2 (SGLT2) inhibitors like for example dapagliflozin, canagliflozin, empagliflozin, ertugliflozin, ipragliflozin, sotagliflozin, luseogliflozin and other diabetes related drug substances like guar gum, repaglinide, nateglinide, pramlintide, benfluorex, mitiglinide and tolrestat.

Vitamins include any vitamin within the groups vitamin A, vitamin B, vitamin C, vitamin D, vitamin E and vitamin K.

Another preferred aspect of the present invention relates to drugs for prophylactic use and/or treatment of disease related to blood and blood forming organs. Such drug substances are typically included in ATC group B. These drug substances include antitrombotic agents including vitamin K antagonists like for example dicoumarol, phenindione and warfarin including heparins, including platelet aggregation inhibitors like for example picotamide, clopidogrel, ticlopidine, acetylsalicylic acid and dipyridamole, direct thrombin inhibitors like for example desirudin, lepirudin, argatroban, melagatran, ximelagatran, bivalirudin and dabigatran etexilate, direct factor Xa inhibitors like for example rivaroxaban, apixaban, edoxaban and betrixaban and other antithrombotic agents.

Another preferred aspect of the present invention relates to drugs for prophylactic use and/or treatment of disease related to the cardiovascular system. Such drug substances are typically included in ATC group B. Drug substances related to the cardiovascular system include cardiac therapy like cardiac glycosides, antiarrhythmics, cardiac stimulants and vasodilators. Drug substances for treatment of hypertension including beta blocking agents like for example metoprolol and atenolol, diuretics like for example hydrochlorothiazide, calcium antagonists like amlodipine and nifedipine, ACE inhibitors like for example enalapril and captopril, angiotensin II receptor antagonists like for example losartan, candesartan and valsartan, lipid modifying agents like for example simvastatin, atorvastatin and ezetimibe.

Another preferred aspect of the present invention relates to drugs for prophylactic use and/or treatment of disease related to skin and include dermatological agents. Such drug substances are typically included in ATC group D.

Another preferred aspect of the present invention relates to drugs for prophylactic use and/or treatment of disease related to the genitourinary system including sex hormones. Such drug substances are typically included in ATC group G. Such drug substances include gynecological antiinfectives and antiseptics for example imidazole derivatives like for example metronidazole, clotrimazole, econazole and ornidazole, triazole derivatives like for example terconazole, antibiotics like natamycin, amphotericin B and candicidin, contraceptives and sex hormones like estrogens, progestogens, androgens and antiandrogens.

Another preferred aspect of the present invention relates to drugs for prophylactic use and/or treatment of disease related to hormones. Such drug substances are typically included in ATC group H. Hormones for systemic use including pituitary and hypoyhalamic hormones, corticosteroids and other hormones in clinical use.

Another preferred aspect of the present invention relates to drugs for prophylactic use and/or treatment of disease related to antiinfectives like antibacterials, antifungal agents and antiviral agents. Such drug substances are typically included in ATC group H.

Antibacterials include drug substances like tetracyclines, chloramphenicol, beta-lactam antibiotics like penicillins and cephalosporines, sulfonamides and trimethoprim, macrolides, lincosamides and strepogramins, aminoglycoside antibacterials, quinolone antibacterials,

Antifungals include substances like for example imidazole derivatives, triazole derivatives, nystatin and amphotericin B.

Antivirals include substances like for example thiosemicarbazones, non-reverse transcriptase inhibitors nucleosides and nucleotides, cyclic amines, phosphonic acid derivatives, protease inhibitors, nucleoside and nucleotide reverse transcriptase inhibitors, non-nucleoside reverse transcriptase inhibitors, neuraminidase inhibitors, integrase inhibitors, antintivirals for treatment of HCV infections.

Another preferred aspect of the present invention relates to drugs for prophylactic use and/or treatment of disease related to antineoplastic drug substances and immunomodulating agents. Such drug substances are included in ATC group L.

Antineoplastic drugs are included in ATC group LI. A preferred aspect of the present invention relates to drugs within ATC group L01.

Antineoplastic drugs include alkylating agents like for example cyclophosphamide, chlorambucil, melphalan, chlormethine, ifosfamide, trofosfamide, prednimustine, bendamustine, busulfan, treosulfan, mannosulfan, thiotepa, triaziquone, carboquone, carmustine, lomustine, semustine, streptozocin, fotemustine, nimustine, ranimustine, uramustine, etoglucid, mitobronitol, pipobroman, temozolomide and dacarbazine, including antimetabolites like for example methotrexate, raltitrexed, pemetrexed, pralatrexate, mercaptopurine, tioguanine, cladribine, fludarabine, clofarabine, nelarabine, cytarabine, fluorouracil, tegafur, carmofur, gemcitamine, capecitabine, azacitidine, decitabine, floxuridine, trifluridine, including plant alkaloids and other natural products like for example vinblastine, vincristine, vindesine, vinorelbine, vinflunine, vintafolide, etoposide, teniposide, demecolcine, paclitaxel, docetaxel, paclitaxel poliglumex, cabazitaxel, topotecan, irinotecan, etirinotecan pegol, belotecan and trabectedin, including cytotoxic antibiotics and related substances like for example dactinomycin, doxorubicin, daunorubicin, epirubicin, aclarubicin, zorubicin, idarubicin, mitoxantrone, pirarubicin, valrubicin, amrubicin, pixantrone, bleomycin, plicamycin mitomycin and ixabepilone, including protein kinase inhibitors like BCR-ABL tyrosine kinase inhibitors for example imatinib, dasatinib, nilotinib, bosutinib and ponatinib, like epidermal growth factor receptor (EGFR) tyrosine kinase inhibitors for example gefitinib, erlotinib, afatinib, osimertinib, rociletinib, olmutinib, dacomitinib and icotinib, like B-Raf serine-threonine kinase (BRAF) inhibitors for example vemurafenib, dabrafenib and encorafenib, like anaplastic lymphoma kinase (ALK) inhibitors for example crizotinib ceritinib, alectinib, brigatinib and lorlatinib, like Mitogen-activated protein kinase (MEK) inhibitors for example trametinib, cobimetinib, binimetinib and selumetinib, like Cyclin-dependent kinase (CDK) inhibitors for example palbociclib, ribociclib and abemaciclib, like mammalian target of rapamycin (mTOR) kinase inhibitors for example temsirolimus everolimus and ridaforolimus, like human epidermal growth factor receptor 2 (HER2) tyrosine kinase inhibitors for example lapatinib, neratinib and tucatinib, like Janus-associated kinase (JAK) inhibitors for example ruxolitinib and fedratinib, like vascular endothelial growth factor receptor (VEGFR) tyrosine kinase inhibitors for example axitinib, cediranib and tivozanib, like Bruton's tyrosine kinase (BTK) inhibitors for example ibrutinib, acalabrutinib and zanubrutinib, like phosphatidylinositol-3-kinase (Pi3K) inhibitors for example idelalisib, copanlisib, alpelisib and duvelisib, like other protein kinase inhibitors for example sunitinib, sorafenib, pazopanib, vandetanib, regorafenib, masitinib, cabozantinib, lenvatinib, nintedanib, midostaurin, quizartinib, larotrectinib, gilteritinib, entrectinib, pexidartinib, erdafitinib, capmatinib, avapritinib, ripretinib, pemigatinib and tepotinib, other antineoplastic agents like platinum compounds for example cisplatin, carboplatin, oxaliplatin, satraplatin and polyplatillen, like methylhydrazines for example procarbazine, like monoclonal antibodies for example edrecolomab, rituximab, trastuzumab, gemtuzumab ozogamicin, cetuximab, bevacizumab, panitumumab, catumaxomab, ofatumumab, ipilimumab, brentuximab vedotin, pertuzumab, trastuzumab emtansine, obinutuzumab, dinutuximab beta, nivolumab, pembrolizumab, blinatumomab, ramucirumab, necitumumab, elotuzumab, daratumumab, mogamulizumab, inotuzumab ozogamicin, olaratumab, durvalumab, bermekimab, avelumab, atezolizumab, cemiplimab, moxetumomab pasudotox, tafasitamab, enfortumab vedotin, polatuzumab vedotin, isatuximab, belantamab mafodotin, dostarlimab and trastuzumab deruxtecan, like sensitizers used in photodynamic/radiation therapy for example porfimer sodium, methyl aminolevulinate, aminolevulinic acid, temoporfin, efaproxiral, padeliporfin, like retinoids for cancer treatment for example tretinoin, alitretinoin and bexarotene, like proteasome inhibitors for example bortezomib, carfilzomib and ixazomib, like histone deacetylase (HDAC) inhibitors for example vorinostat, romidepsin, panobinostat, belinostat and entinostat, like hedgehog pathway inhibitors for example vismodegib, sonidegib and glasdegib, like poly (ADP-ribose) polymerase (PARP) inhibitors for example olaparib, niraparib, rucaparib, talazoparib and veliparib, like other antineoplastic agents for example amsacrine, asparaginase, altretamine, hydroxycarbamide, lonidamine, pentostatin, masoprocol, estramustine, mitoguazone, tiazofurine, mitotane, pegaspargase, arsenic trioxide, denileukin diftitox, celecoxib, anagrelide, oblimersen, sitimagene ceradenovec, omacetaxine mepesuccinate, eribulin, aflibercept, talimogene laherparepvec, venetoclax, vosaroxin, plitidepsin, epacadostat, enasidenib, ivosidenib, selinexor, tagraxofusp, lurbinectedin, axicabtagene ciloleucel and tisagenlecleucel.

Drug substances for endocrine therapy including hormones and antihormones. These drug substances are included in ATC group L02.

Immunostimulant are included in ATC group L03. A preferred aspect of the present invention relates to drugs within ATC group L03. Immunostimulants include colony stimulating factors for example filgrastim, molgramostim, sargramostim, lenograstim,
ancestim, pegfilgrastim, lipegfilgrastim, balugrastim, empegfilgrastim, and pegteograstim, including interferons for example interferon alfa natural, interferon beta natural, interferon gamma, interferon alfa-2a, interferon alfa-2b, interferon alfa-n1, interferon beta-1a, interferon beta-1b, interferon alfacon-1, peginterferon alfa-2b, peginterferon alfa-2a, albinterferon alfa-2b, peginterferon beta-1a, cepeginterferon alfa-2b, ropeginterferon alfa-2b, including interleukins for example aldesleukin and oprelvekin, including other immunostimulants for example lentinan, roquinimex, BCG vaccine, pegademase, pidotimod, poly I:C, polyICLC, thymopentin, immunocyanin, tasonermin, melanoma vaccine, glatiramer acetate, histamine, mifamurtide, plerixafor, sipuleucel-T, cridanimod, dasiprotimut-T and elapegademase

Immunosuppressants are included in ATC group L04. A preferred aspect of the present invention relates to drugs within ATC group L04. Immunosuppressants including selective immunosuppressants for example muromonab-CD3, antilymphocyte immunoglobulin (horse), antithymocyte immunoglobulin (rabbit), mycophenolic acid including mycophenolate mofetil, sirolimus, leflunomide, alefacept, everolimus, gusperimus, efalizumab, abetimus, natalizumab, abatacept, eculizumab, belimumab, fingolimod, belatacept, tofacitinib, teriflunomide, apremilast, vedolizumab, alemtuzumab, begelomab, ocrelizumab, baricitinib, ozanimod, emapalumab, cladribine, imlifidase, siponimod, ravulizumab, upadacitinib, filgotinib, itacitinib, inebilizumab, including tumor necrosis factor alpha (TNF-alpha) inhibitors for example etanercept infliximab, afelimomab, adalimumab, certolizumab pegol, golimumab and opinercept, including interleukin inhibitors for example daclizumab, basiliximab, anakinra, rilonacept, ustekinumab, tocilizumab, canakinumab, briakinumab, secukinumab, siltuximab, brodalumab, ixekizumab, sarilumab, sirukumab, guselkumab, tildrakizumab, risankizumab and satralizumab, including calcineurin inhibitors for example ciclosporin, tacrolimus and voclosporin including other immunosuppressants for example azathioprine, thalidomide, methotrexate, lenalidomide, pirfenidone, pomalidomide, dimethyl fumarate and darvadstrocel.

Another preferred aspect of the present invention relates to drugs for prophylactic use and/or treatment of disease related to muscular and skeletal system including anti-inflammatory and antirheumatic compounds and immunmodulating agents. Such drug substances are included in ATC group M. Drug substances related to muscular and skeletal system including anti-inflammatory and antirheumatic compounds for example non-steroid anti-inflammatory compounds including for example indomethacin, diclofenac, ibuprofen and naproxen, and muscle relaxants.

Another preferred aspect of the present invention relates to drugs for prophylactic use and/or treatment of disease related to the nerve system. Such drug substances are included in ATC group N. Drug substances related to the nerve system include anesthetics, analgesics, antiepileptics, anti-parkinson drug substances, psycholeptics, psychoanaleptics and other drug substances with effect on the nervous system. Some examples of drug substances and groups of drug substances related to the nervous system include opioids like for example natural opium alkaloids like morphine, codeine, and oxycodone and synthetic compounds like pethidine, ketobemidone and fentanyl, antiepileptics like for example barbiturates, hydantoin derivatives, oxazolidine derivatives, succinimide derivatives, benzodiazepine derivatives, carboxamide derivatives and fatty acid derivatives, antiparkinson drugs like anticholinergic agents and dopaminergic agents, phycoleptics like antipsychotics, anxiolytics and hypnotics and sedatives, psychoanaleptics like antidepressants, psychostimulants, drug substances used for ADHD, nootropics, psycholeptics and anti-dementia drugs.

Another preferred aspect of the present invention relates to drugs for prophylactic use and/or treatment of disease related to the respiratory system. Such drug substances are included in ATC group R. Drug substances related to the respiratory system include nasal compositions, throat compositions, drugs for treatment of obstructive pulmonary diseases like asthma and COPD, cough and cold compositions and antihistamines.

Another preferred aspect of the present invention relates to drugs for prophylactic use and/or treatment of disease for use in ear and eye. Such drug substances are included in ATC group S.

In a particularly preferred embodiment, the at least one drug substance is selected from the group consisting of atorvastatin, simvastatin, losartan, valsartan, candesartan, enalapril, atenolol, propranolol, hydrochlotiazide, cyclosporine, amphotericin B, dilthiazem, phenoxymethylpenicillin, azithromycin, rapamycin, griseofulvin, chloramphenicol, erythromycin, acyclovir, nystatin, phenytoin, phenobarbital, ampicillin, celecoxib, prednisolon and metformin

A highly preferred embodiment of the present invention relates to cCVD-SP comprising one or more drug substance wherein said drug substance is poorly soluble in water.

In a particularly preferred embodiment of the invention, the one or more drug substance(s) is in the form of a complex with a cyclodextrin.

The most frequently used drug complexes in clinical use are complexes with cyclodextrins. Cyclodextrins are cyclic oligosaccharides comprising 6-8 glucose subunits. α (alpha)-Cyclodextrin comprises of 6 glucose subunits, β (beta)-cyclodextrin comprises of 7 glucose subunits and γ (gamma)-cyclodextrin comprised of 8 glucose subunits. Any cyclodextrin or derivative thereof can be used in the present invention. The most preferred cyclodextrins are beta-cyclodextrin, 2-hydroxypropyl-beta-cyclodextrin and 4-sulphobutyl-beta-cyclodextrin.

A preferred embodiment of the present invention relates to cCVD-SP comprising one drug substance wherein said drug substance is in the form of a complex with a cyclodextrin.

A more preferred embodiment of this aspect the present invention relates to cCVD-SP comprising one drug substance where said drug substance is in the form of a complex with a beta-cyclodextrin or derivatives thereof.

Even more preferred embodiment of this aspect the present invention relates to PcCVD-SP comprising one drug substance are in the form of a complex with a cyclodextrin beta-cyclodextrin or derivatives thereof.

A preferred embodiment of the present invention relates to cCVD-SP comprising two drug substances where at least one said drug substance is in the form of a complex with a cyclodextrin.

A more preferred embodiment of this aspect the present invention relates to PcCVD-SP comprising two drug substances where at least one said drug substance is in the form of a complex with a beta-cyclodextrin or derivatives thereof.

A preferred embodiment of the present invention relates to cCVD-SP comprising three or more drug substances where at least one said drug substance is in the form of a complex with a cyclodextrin.

A more preferred embodiment of this aspect the present invention relates to PcCVD-SP comprising three or more drug substances where at least one said drug substance is in the form of a complex with a beta-cyclodextrin or derivatives thereof.

The invention further related to methods for the production of cCVD-SP or PcCVD-SP loaded with at least one drug cyclodextrin complex characterized by mixing cCVD-SP or PcCVD-SP with at least one drug cyclodextrin complex at ambient temperature in a solvent where the particles are dispersed and drug cyclodextrin complex is, at least partly, soluble.

Another preferred method for production of cCVD-SP or PcCVD-SP loaded with at least one drug cyclodextrin complex is characterized by mixing the cCVD-SP or PcCVD-SP with cyclodextrin at ambient temperature in a solvent where the particles are dispersed and drug cyclodextrin complex is, at least partly, soluble, optionally followed by isolation of the particles, followed by generation of the drug cyclodextrin complex within the particles by mixing the cCVD-SP or PcCVD-SP with a drug substance in a solvent where the particles are dispersed and drug substance is, at least partly, soluble.

The silicon particles of the invention preferably comprise the at least one drug substance in an amount of 5 to 50 wt %, more preferably 15 to 40 wt %, relative to the total weight of the silicon particles. Where more than one drug substance is present, it will be understood that these wt % ranges refer to the combined wt % of all drug substances present. Furthermore, where one or more of the drug substances is in the form of a cyclodextrin complex, the above quoted wt % ranges are to be based on to the total weight of the cyclodextrin complex.

### Compositions and Uses

The present invention further relates to pharmaceutical compositions comprising silicon particles as hereinbefore defined and one or more pharmaceutically acceptable carriers, diluents or excipients. Such carriers, diluents and excipients are well known in the art.

Excipients used in the pharmaceutical compositions of the present invention will vary depending on the nature of the composition. Excipients for suspensions of cCVD-SP or PcCVD-SP are, in addition to water, typically selected among sodium chloride or other physiologically acceptable salts, sugars, surfactant, antioxidants aromas, sweeteners and pH modifiers.

The silicon particles and compositions thereof may be used in therapy, in particular in drug delivery. Hence, the present invention relates to silicon particles according to the present invention for use in therapy. In a further embodiment, the present invention relates to the silicon particles according to the current invention for use in the treatment or prevention, or the diagnosis of particular disorders and diseases. Examples of disorders or diseases which can be treated or prevented in accordance with the present invention include cancer, such as lung cancer, breast cancer, prostate cancer, head and neck cancer, ovarian cancer, skin cancer, testicular cancer, pancreatic cancer, colorectal cancer, kidney cancer, cervical cancer, gastrointestinal cancer and combinations thereof; pain related diseases; diabetes; hypertension and immune related diseases.

The nanoparticles or compositions thereof are preferably administered in a therapeutically effective amount. A “therapeutically effective amount” refers to an amount of the nanoparticles necessary to treat or prevent the particular disease or disorder. Any route of administration may be used to deliver the nanoparticles to the subject. Suitable administration routes include intramuscular injection, transdermal administration, inhalation, topical application, oral administration, rectal or vaginal administration, intertumural administration and parenteral administration (e.g. intravenous, peritoneal, intra-arterial or subcutaneous). The preferable route of administration is oral or subcutaneous.

For oral administration aqueous suspension, tablet and capsules are the most preferred formulations, for dermal use creams and ointments are preferred pharmaceutical formulations. Regarding injections, the most preferred injections are intravenous injections, intramuscular injections and subcutaneous injections. The injection formulations are typically in the form of sterile aqueous suspensions. Pulmonary formulations according the present invention in the form of dry powder for inhalation, are typically in the form of single doses or multi dose, or in the form of suspension of particles. Eye products are typically sterile aqueous suspensions of particles, while typical compositions for administration into the nose can be dry particles or an aqueous suspension.

Typically oral capsules comprising cCVD-SP or PcCVD-SP are capsules prepared from gelatin or hydroxypropyl methyl cellulose (HPMC). Typical excipients in such capsules might include lactose, microcrystalline cellulose and inorganic salts.

Typically tablets comprising cCVD-SP or PcCVD-SP can be tablets that disintegrate immediately, controlled release tablets and sustained release tablets. Typical excipients in tablets include for example corn starch, lactose, glucose, microcrystalline cellulose, croscarmellose sodium and magnesium stearate.

The exact dosage and frequency of administration depends on the particular nanoparticles, active agent and targeting agents used, the particular condition being treated, the severity of the condition being treated, the age, weight, sex, extent of disorder and general physical condition of the particular patient as well as other medication the individual may be taking, as is well known to those skilled in the art. Furthermore, it is evident that said effective daily amount may be lowered or increased depending on the response of the treated subject and/or depending on the evaluation of the physician prescribing the nanoparticles according to the instant invention.

One embodiment of the present invention relates to pharmaceutical compositions comprising drug-loaded cCVD-SP or PcCVD-SP. The pharmaceutical composition can be in any pharmaceutically acceptable formulation depending on route of administration. For oral administration aqueous suspension, tablet and capsules are the most preferred formulations, for dermal use creams and ointments are preferred pharmaceutical formulations. Regarding injections, the most preferred injections are intravenous injections, intramuscular injections and subcutaneous injections. The injection formulations are typically in the form of sterile aqueous suspensions. Pulmonary formulations according the present invention in the form of dry powder for inhalation, are typically in the form of single doses or multi dose, or in the form of suspension of particles. Eye products are typically sterile aqueous suspensions of particles, while typical compositions for administration into the nose can be dry particles or an aqueous suspension.

In one embodiment, the pharmaceutical compositions as hereinbefore described are formulation for parenteral administration, e.g. injection or infusion.

One preferred embodiment of this aspect of the invention relates to pharmaceutical compositions comprising cCVD-SP or PcCVD-SP.

A more preferred embodiment of this aspect of the invention relates to pharmaceutical compositions comprising cCVD-SP or PcCVD-SP comprising at least one drug substance in the form of free drug substance or pharmaceutically acceptable salts thereof.

An even more preferred embodiment of this aspect of the invention is wherein the pharmaceutical compositions comprise cCVD-SP or PcCVD-SP comprising at least one drug substance in the form of free drug substance or pharmaceutically acceptable salts thereof where said silicon is in an amorphous form.

An even more preferred embodiment of this aspect of the invention is wherein the pharmaceutical compositions comprise cCVD-SP comprising at least one drug substance in the form of free drug substance or pharmaceutically acceptable salts thereof where said silicon is in an amorphous form.

Another even more preferred embodiment of this aspect of the invention is wherein the pharmaceutical compositions comprise non-etched cPCVD-SP comprising at least one drug substance in the form of free drug substance or pharmaceutically acceptable salts thereof where said silicon is in an amorphous form.

Another preferred embodiment of this aspect of the invention relates to pharmaceutical compositions comprising cCVD-SP or PcCVD-SP comprising at least one drug substance in the form of cyclodextrin complex.

An even more preferred embodiment of this aspect of the invention relates to pharmaceutical compositions comprising cCVD-SP or PcCVD-SP comprising at least one drug substance in the form of cyclodextrin complex wherein said silicon is in an amorphous form.

A further preferred embodiment of this aspect of the invention relates to pharmaceutical compositions comprising cCVD-SP or PcCVD-SP comprising at least one drug substance in the form of cyclodextrin complex wherein said silicon is in crystalline form.

An even more preferred embodiment of this aspect of the invention is wherein the pharmaceutical compositions comprise cCVD-SP comprising at least one drug substance in the form of cyclodextrin complex where said silicon is in an amorphous form.

Another preferred embodiment of this aspect of the invention is wherein the pharmaceutical compositions comprise cCVD-SP comprising at least one drug substance in the form of cyclodextrin complex where said silicon is in crystalline form.

An even more preferred embodiment of this aspect of the invention is wherein the pharmaceutical compositions comprise cCVD-SP or PcCVD-SP comprising at least one drug substance in the form of beta-cyclodextrin complex where said silicon is in an amorphous form.

A further preferred embodiment of this aspect of the invention is wherein the pharmaceutical compositions comprise cCVD-SP or PcCVD-SP comprising at least one drug substance in the form of beta-cyclodextrin complex where said silicon is in crystalline form.

In one particularly preferred embodiment of the invention, the pharmaceutical compositions as hereinbefore defined are formulated for oral administration, e.g. as tablets, capsules or a suspension.

A more preferred embodiment of this aspect of the invention is wherein the pharmaceutical compositions comprise cCVD-SP or PcCVD-SP comprising at least one drug substance in the form of free drug substance.

An even more preferred embodiment of this aspect of the invention is wherein the pharmaceutical compositions comprise cCVD-SP or PcCVD-SP comprising at least one drug substance in the form of free drug substance where said silicon is in an amorphous form.

An even more preferred embodiment of this aspect of the invention is wherein the pharmaceutical compositions comprise cCVD-SP comprising at least one drug substance in the form of free drug substance where said silicon is in an amorphous form.

Another even more preferred embodiment of this aspect of the invention is wherein the pharmaceutical compositions comprise non-etched cPCVD-SP comprising at least one drug substance in the form of free drug substance where said silicon is in an amorphous form.

Another more preferred embodiment of this aspect of the invention is wherein the pharmaceutical compositions comprise cCVD-SP or PcCVD-SP comprising at least one drug substance in the form of cyclodextrin complex.

An even more preferred embodiment of this aspect of the invention is wherein the pharmaceutical compositions comprise cCVD-SP or PcCVD-SP comprising at least one drug substance in the form of cyclodextrin complex wherein said silicon is in an amorphous form.

An even more preferred embodiment of this aspect of the invention is wherein the pharmaceutical compositions comprise cCVD-SP comprising at least one drug substance in the form of cyclodextrin complex where said silicon is in an amorphous form.

An even more preferred embodiment of this aspect of the invention is wherein the pharmaceutical compositions comprise cCVD-SP or PcCVD-SP comprising at least one drug substance in the form of beta-cyclodextrin complex where said silicon is in an amorphous form.

A further preferred embodiment of this aspect of the invention is wherein the pharmaceutical compositions comprise cCVD-SP comprising at least one drug substance in the form of unsubstituted beta-cyclodextrin complex.

A further preferred embodiment of this aspect of the invention is wherein the pharmaceutical compositions comprise non-etched PcCVD-SP comprising at least one drug substance in the form of unsubstituted beta-cyclodextrin complex.

An even more preferred aspect of this aspect of the invention is wherein the pharmaceutical compositions comprise cCVD-SP comprising at least one drug substance in the form of unsubstituted beta-cyclodextrin complex, 2-hydroxypropyl-beta-cyclodextrin complex or 4-sulphobutyl-beta-cyclodextrin complex.

An even more preferred aspect of this aspect of the invention is wherein the pharmaceutical compositions comprise non-etched PcCVD-SP comprising at least one drug substance in the form of unsubstituted beta-cyclodextrin complex, 2-hydroxypropyl-beta-cyclodextrin complex or 4-sulphobutyl-beta-cyclodextrin complex.

Most embodiment of this aspect of the invention is wherein the pharmaceutical compositions comprise cCVD-SP comprising at least one drug substance in the form of unsubstituted beta-cyclodextrin complex, 2-hydroxypropyl-beta-cyclodextrin complex or 4-sulphobutyl-beta-cyclodextrin complex.

The BCS (Biopharmaceutics Classification System) is a system to differentiate the drugs on the basis of their aqueous solubility and oral permeability, BCS Class II drug substances are compounds with low water solubility but high oral permeability. See for example Amidon G L, Lennernäs H, Shah V P, Crison J R (March 1995). “A theoretical basis for a biopharmaceutic drug classification: the correlation of in vitro drug product dissolution and in vivo bioavailability”. Pharm. Res. 12 (3): 413-20.

A highly preferred embodiment of this aspect of the present invention relates to pharmaceutical compositions of cCVD-SP comprising drug substances where said drug substances are classified as BCS Class II

A further highly preferred embodiment of this aspect of the present invention relates to pharmaceutical compositions of non-etched PcCVD-SP comprising drug substances where said drug substances are classified as BCS Class II drug substances.

The oral bioavailability of drug substances varies from almost 0% to almost 100%. The absolute bioavailability of some of the more frequently used drugs are: atorvastatin (bioavailability 12%), simvastatin (bioavailability less than 5%), losartan (bioavailability 33%), valsartan (bioavailability 25%), candesartan (bioavailability 40%), enalapril (bioavailability 60%), atenolol (bioavailability 40-50%), propranolol (bioavailability 26%), hydrochlotiazide (bioavailability 70%), cyclosporine (bioavailability very low), amphotericin B (bioavailability very low), dilthiazem (bioavailability 40%), phenoxymethylpenicillin (bioavailability 50%), azithromycin (bioavailability 40%), metformin (bioavailability 50-60%).

In the context of pharmaceutical compositions formulation for oral administration, the following represent preferable embodiments.

Another highly preferred embodiment of the present invention relates to pharmaceutical compositions of cCVD-SP comprising drug substances where said drug substances are drug substances with low oral bioavailability per se. Typical low bioavailability is less than 50%, more preferably less than 30%, more preferably less than 20%, most preferably less than 10%.

A highly preferred embodiment of this aspect of the present invention relates to pharmaceutical compositions of PcCVD-SP comprising drug substances where said drug substances are drug substances with low bioavailability per se. Typical low bioavailability is less than 50%, more preferably less than 30%, more preferably less than 20%, most preferably less than 10%.

Another highly preferred embodiment of the present invention relates to pharmaceutical compositions of cCVD-SP comprising drug substances with very low aqueous solubility. Typical very low solubility is less than 100 mg per liter, more preferably less than 50 mg per liter, even more preferably less than 10 mg per liter, most preferably less than 5 mg per liter.

Another highly preferred embodiment of this aspect of the present invention relates to pharmaceutical compositions of PcCVD-SP comprising drug substances with very low aqueous solubility. Typical very low solubility is less than 100 mg per liter, more preferably less than 50 mg per liter, even more preferably less than 10 mg per liter, most preferably less than 5 mg per liter.

The list of drugs that are almost insoluble or have very low aqueous solubility is extensive. A few examples on these well-known drugs in clinical use worldwide include, but are not limited to, simvastatin, lovastatin, celecoxib, naproxen, ibuprofen, estradiol, testosterone, finasteride, glipizide, ketoconazole, methylprednisolone, mometrasone, triamcinolone, griseofulvin and amphotericin B.

Another highly preferred embodiment of the present invention relates to pharmaceutical compositions of cCVD-SP comprising drug substances with partition coefficient value (amount of substance dissolving in water versus organic phase, giving a measure of hydrophobic/hydrophilic properties), log P, above 2.5, more preferably more than 3.0, even more preferably more than 3.5, even more preferably more than 4.0 and most preferably more than 4.5.

Another highly preferred embodiment of this aspect of the present invention relates to pharmaceutical compositions of PcCVD-SP comprising drug substances with log P above 2.5, more preferably more than 3.0, even more preferably more than 3.5, even more preferably more than 4.0 and most preferably more than 4.5.

Some typical examples of well-known drugs in clinical use include the following drug substances with known high log P values are: amiodarone (log P 7.81), amitriptyline (log P 4.41), amlodipine (log P 3.01), antazoline (log P 3.58), ariprazole (log P 3.76), atomoxetine (log P 3.36), bacampicillin (log P 3.52), benzphetamine (log P 3.84), benztropine (log P 4.04), bitolterol (log P 4.16), bosentan (log P 4.36), bromodiphenhydramine (log P 4.03), brompheniramine (log P 3.24), bufuralol (log P 3.54), bupivacaine (log P 3.31), butacaine (log P 4.62), butaclamol (log P 3.81), butorphanol (log P 3.54), carbenoxolone (log P 6.63), carvedilol (log P 4.11), chlorocyclizine (log P 3.24), chlorpromazine (log P 5.35), chlorprothixene (log P 5.31), cinchonine (log P 3.69), citalopram (log P 3.47), clofibrate (log P 3.88), clopenthixol (log P3.91), clotrimazole 4.92), clozapine (log P 3.94), cyclazocine (log P 3.52), cyclobenzaprine (log P 6.19), cyproheptadine (log P 4.92), darifenacin (log P 3.78), deserpidine (log P 4.95), desipramine (log P 3.97), desloratadine (log P 3.50), dextrobrompheniramine (log P 3.24), dextrofenfluramine (log P 3.55), dextromethorphan (log P 3.89), dibenzepin (log P 3.26), dibucaine (log P 4.40), diclofenac (log P 4.55), dicloxacillin (log P 3.10), dicyclomine (log P 4.64), diethazine (log P 5.55), diflunisal (log P 3.65), dihydroergocriptine (log P 6.37), dihydroergocristine (log P 6.55), dihydroergotamine (log P 5.69), dilevalol (log P 3.09), diltiazem (log P 4.73), dimethisoquin (log P 4.04), diperodon (log P 4.65), diphenhydramine (log P 3.27), diphenoxin (log P 3.97), diphenoxylate (log P 4.5 1), diphenylpyraline (log P 3.43), dipipanone (log P 5.10), dipyridamole (log P 3.35), donepezil (log P 3.91), doxepin (log P 3.85), droperidol (log P 3.10), duloxetine (log P 4.81), Emetine (log P 3.82), enalapril (log P 3.25), enalaprilat (log P 3.63), entacapone (log P 3.02), ergotamine (log P 7.37), estrone (log P 3.62), ethopropazine (log 4.77), etidocaine (log P 3.57), etomidate (log P 3.05), fenclofenac (log P 4.59), fenfluramine (log P 3.55), fenoprofen (log P 3.72), fentanyl (log P 3.68), fesoterodine (log P 5.08), fexofenadine (log P 3.73), finasteride (log P 3.83), flurbiprofen (log P 3.66), flufenamic acid (log P 5.22), flumizole (log P 4.26), fluoxetine (log P 3.93), flupenthixol (log P 3.67), fluphenazine enanthate (log P 7.29) fluphenazine (log P 3.92), flurazepam (log P 4.84), flutamide (log P 3.52), fusidic acid (log P 5.76), fluvoxamine (log P 3.71), glibenclamide (log P 3.08), glyburide (log P 3.08), haloperidol (log P 3.76), hexylcaine (log P 3.65), hycanthone (log P 3.81), ibuprofen (log P 3.50), imipramine (log P 4.35), indacaterol (log P 3.88), indomethacin (log P 4.25), iocetamic acid (log P 4.57), iodipamide (log P 5.10), iodoquinol (log P 4.10), iopanoic acid (log P 4.65), iprindole (log P 5.02), irbesartan (log P 5.25), ketamine (log P 3.01), ketoconazole (log P 4.04), levallorphan (Log P 3.85), leverphanol (log P 3.26), liothyronine (log P 3.91), Lisinopril (log P 3.47), loperamide (log P 4.15), loratadine (log P 3.90), losartan (log P 3.46), maprotiline (log P 4.36), meclizine (log P 5.28), meclofenamic acid (log P 5.44), medazepam (log P 3.89), mefenamic acid (log P 4.83), mepazine (log P 5.04), methadone (log P 3.93), methdilazine (log P 4.64), methotrimeprazine (log P 4.94), metolazone (log P 3.16), miconazole (log P 4.97), midazolam (log P 3.80), montelukast (log P 5.81), nabilone (log P 7.25), nebivolol (log P 4.08), nelfinavir (log P 7.28), nortriptyline (log P 3.97), novobiocin (log P 3.74), olanzapine (log P 3.08), orphenadrine (log P 3.33), oxybutynin (log P 5.05), oxyphenylbutazone (log P 3.28), pamaquine (log P 4.38), penbutolol (log P 4.02), pentazocine (log P 4.15), pergolide (log P 3.90), perphenazine (log P 3.94), perhexilene (log P 6.46), phencyclidine (log P 4.25), phenindamine (log P 3.81), phenindione (log P 3.19), phenothiazine (log P 4.15), phenoxybenzamine (log P 3.69), phentolamine (log P 4.08), phenylbutazone (log P 3.38), phenyltoloxamine (log P 3.46), pimozide (log P 5.57), pipradrol (log P 3.61), pivampicillin (log P 3.88), prasugel (log P 4.31), prazepam (log P 3.70), prochlorperazine (log P 4.65), promazine (log P 4.69), promethazine (log P 4.89), proparacine (log P 3.46), propoxyphene 8 Log P 4.10), pyrathiazine (log P 4.15), pyrrobutamine (log P 4.57), quinacrine (log P 5.59), risperidone (log P 3.04), reserpine (log P 3.65), salmeterol (log P 3.71), salsalate (log P 3.29), sertraline (log P 5.08), solifenacin (log P 3.70), spiperone (log P 3.25), sufentanil (log P 3.95), sulfasalazine (log P 3.05), tamoxifen (log P 5.13), tetracaine (log P 3.75), tetrahydrocannabinol (log P 6.84), thiopropazate (log P 4.76), thioridazine (log P 5.90), thiothixene (log P 3.72), L-thyrosine (log P 4.72), tiagabine (log P 4.03), ticrynfen (log P 3.05), tolterodine (log P 5.23), trifluoperazine (log P 4.62), triflupromazine (log P 5.16), trimeprazine (log P 5.04), trimipramine (log P 4.71), triprolidine (log P 3.25), troleandomycin (log P 3.46), vardenafil (log P 3.64), valsartan (log P 4.02), verapamil (log P 4.02), vinblastine (log P 5.92), vincristine (log P 5.75) vindesine (log P 4.94), warfarin (log P 3.13) and zimeldine (log P 3.07). All log P values are calculated log P values from Foye's Principles of Medicinal Chemistry. (Thomas L. Lemke, David A. Williams, Victoria F. Roche and S. William Zito), Seventh Edition, Lippincott Williams&Wilkins (2011).

FIG. 1: Silicon particles of average diameter 300 nm produced by cCVD

FIG. 2: Silicon articles with primary particle size (median average diameter) is about 30 nm. Dynamic light scattering (DLS) size of these particles is about 150 nm and BET analysis gives an average particle size of about 40 nm.

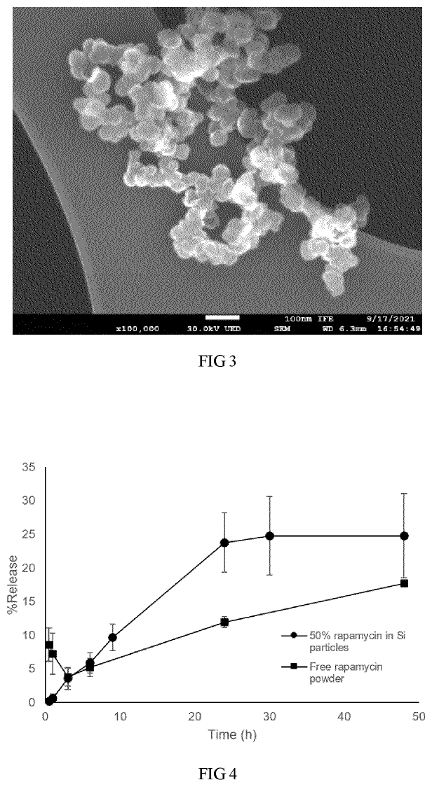

FIG. 3: SEM Image of amorphous aggregated cCVD Si particles

FIG. 4: Rapamycin release vs. time for Example 47

FIG. 5: Rapamycin release vs. time for Example 48

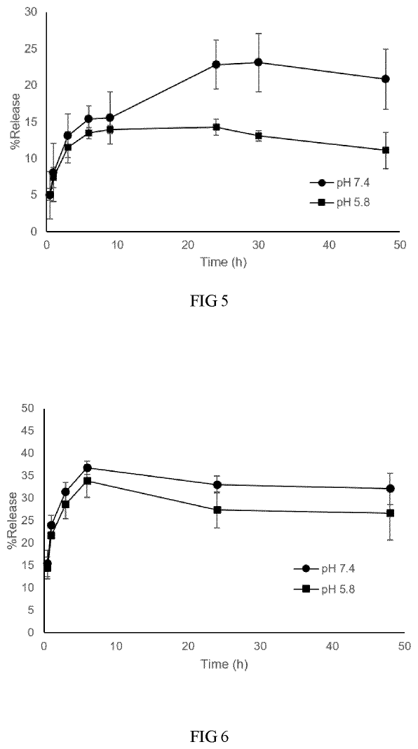

FIG. 6: Rapamycin release vs. time for Example 49

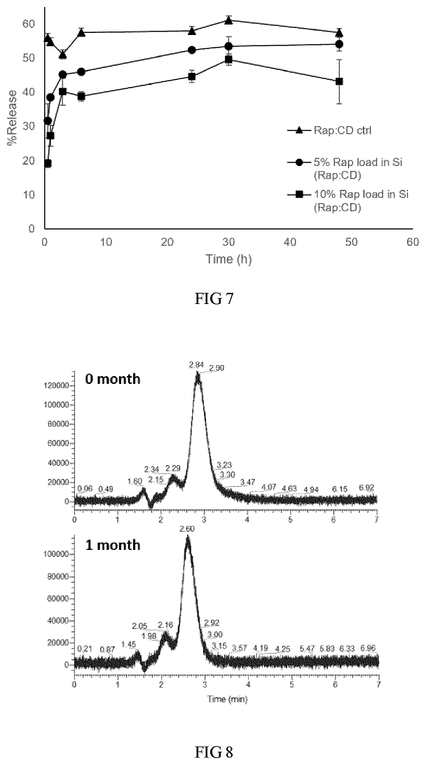

FIG. 7: Rapamycin release vs. time for Example 50 and 51

FIG. 8: HPLC analysis/chromatogram for identification of rapamycin, and no degradation peaks, in Example 52

The invention will now be described with reference to the following, non-limiting, examples.

### EXAMPLES

All silicon particles were produced by CVD in a reactor where the reactor comprise a reactor body and a rotation device operatively arranged to the reactor, wherein the rotation device is configured to rotate the reactor around an axis during production according to WO2013048258.

When used for preparation of loaded silicon particles the mortar and pestle were cleaned in 2 M sodium hydroxide and washed with water before preparation of new batches of silicon particles.

PDI is polydispersity index

All drug release experiments, except rapamycin experiments, were performed with excess drug substance in purified water with samples rolling on a rolling table at room temperature.

The samples for HPLC analysis were centrifugated for 30 minutes at 13 000 rpm. before further sample preparations.

### Intermediate 1 Atorvastatin Calcium 2-Hydroxypropyl-Beta-Cyclodextrin Complex (1:4.2)

Atorvastatin calcium (DDL, 559 mg, 0.48 mmol) and 2-hydroxy-propyl-beta-cyclodextrin (Aldrich, mw 1380, 2.76 g, 2 mmol) were volumetrically mixed in a mortar. A mixture of water/ethanol (1:1 (v/v)) was added to obtain a viscous paste using mortar and pestle. The paste was mixed for 5 minutes and dried over night at 50 degrees centigrade. A white powder comprising 17% (w/w) atorvastatin calcium was isolated.

### Intermediate 2 Griseofulvin 2-Hydroxypropyl-Beta-Cyclodextrin Complex (1:3)

Griseofulvin (DDL, 352 mg, 1 mmol) and 2-hydroxy-propyl-beta-cyclodextrin (Biosynth Carbosynth, 4.38 g, 3 mmol) were volumetrically mixed in a mortar. Water was added to obtain a viscous paste using mortar and pestle. The paste was mixed for 5 minutes and dried over night at 60 degrees centigrade. A white powder comprising 7.4% (w/w) griseofulvin was isolated.

### Intermediate 3 Chloramphenicol 2-Hydroxypropyl-Beta-Cyclodextrin Complex (1:3)

Chloramphenicol (DDL, 323 mg, 1 mmol) and 2-hydroxy-propyl-beta-cyclodextrin (Biosynth Carbosynth, 4.38 g, 3 mmol) were volumetrically mixed in a mortar. Water was added to obtain a viscous paste using mortar and pestle. The paste was mixed for 5 minutes and dried over night at 60 degrees centigrade. A white powder comprising 6.9% (w/w) chloramphenicol was isolated.

### Intermediate 4 Erythromycin 2-Hydroxypropyl-Beta-Cyclodextrin Complex (1:3)

Erythromycin (DDL, 733 mg, 1 mmol) and 2-hydroxy-propyl-beta-cyclodextrin (Biosynth Carbosynth, 4.38 g, 3 mmol) were volumetrically mixed in a mortar. Water was added to obtain a viscous paste using mortar and pestle. The paste was mixed for 5 minutes and dried over night at 60 degrees centigrade. A white powder comprising 14.3% (w/w) erythromycin was isolated.

### Intermediate 5 Losartan Potassium 2-Hydroxypropyl-Beta-Cyclodextrin Complex (1:3)

Losartan potassium (DDL, 461 mg, 1 mmol) and 2-hydroxy-propyl-beta-cyclodextrin (Biosynth Carbosynth, 4.38 g, 3 mmol) were volumetrically mixed in a mortar. Water was added to obtain a viscous paste using mortar and pestle. The paste was mixed for 5 minutes and dried over night at 60 degrees centigrade. A white powder comprising 9.5% (w/w) losartan potassium was isolated.

### Intermediate 6 Atorvastatin Calcium 2-Hydroxypropyl-Beta-Cyclodextrin Complex (1:3)

Atorvastatin calcium (DDL, 461 mg, 1 mmol) and 2-hydroxy-propyl-beta-cyclodextrin (Biosynth Carbosynth, 4.38 g, 3 mmol) were volumetrically mixed in a mortar. Water was added to obtain a viscous paste using mortar and pestle. The paste was mixed for 5 minutes and dried over night at 60 degrees centigrade. A white powder comprising 20.9% (w/w) atorvastatin calcium was isolated.

### Intermediate 7 Aciclovir 2-Hydroxypropyl-Beta-Cyclodextrin Complex (1:1)

Aciclovir (DDL, 225 mg, 1 mmol) and 2-hydroxy-propyl-beta-cyclodextrin (Biosynth Carbosynth, 1.46 g, 1 mmol) were volumetrically mixed in a mortar. Water was added to obtain a viscous paste using mortar and pestle. The paste was mixed for 5 minutes and dried over night at 60 degrees centigrade. A white powder comprising 13.4.% (w/w) aciclovir was isolated.

### Intermediate 8 Nystatin 2-Hydroxypropyl-Beta-Cyclodextrin Complex (1:1)

Nystatin (DDL, 773 mg, 1 mmol) and 2-hydroxy-propyl-beta-cyclodextrin (Biosynth Carbosynth, 1.46 g, 1 mmol) were volumetrically mixed in a mortar. Water was added to obtain a viscous paste using mortar and pestle. The paste was mixed for 5 minutes and dried over night at 60 degrees centigrade. A white powder comprising 32.4.% (w/w) nystatin was isolated.

### Intermediate 9 Celecoxib 2-Hydroxypropyl-Beta-Cyclodextrin Complex (1:1)

Celecoxib (DDL, 381 mg, 1 mmol) and 2-hydroxy-propyl-beta-cyclodextrin (Biosynth Carbosynth, 1.46 g, 1 mmol) were volumetrically mixed in a mortar. Water was added to obtain a viscous paste using mortar and pestle. The paste was mixed for 5 minutes and dried over night at 60 degrees centigrade. A white powder comprising 20.7% (w/w) celecoxib was isolated.

### Intermediate 10 Erythromycin 2-Hydroxypropyl-Beta-Cyclodextrin Complex (1:1)

Erythromycin (DDL, 733 mg, 1 mmol) and 2-hydroxy-propyl-beta-cyclodextrin (Biosynth Carbosynth, 1.46 g, 1 mmol) were volumetrically mixed in a mortar. Water was added to obtain a viscous paste using mortar and pestle. The paste was mixed for 5 minutes and dried over night at 60 degrees centigrade. A white powder comprising 33.4% (w/w) erythromycin was isolated.

### Intermediate 11 Griseofulvin 2-Hydroxypropyl-Beta-Cyclodextrin Complex (1:1)

Griseofulvin (Sigma Aldrich, 352 mg, 1 mmol) and 2-hydroxy-propyl-beta-cyclodextrin (Biosynth Carbosynth, 1.46 g, 1 mmol) were volumetrically mixed in a mortar. Water was added to obtain a viscous paste using mortar and pestle. The paste was mixed for 5 minutes and dried over night at 60 degrees centigrade. A white powder comprising 19.4% (w/w) griseofulvin was isolated.

### Intermediate 12 Griseofulvin 2-Hydroxypropyl-Beta-Cyclodextrin Complex (1:2)

Griseofulvin (Sigma Aldrich, 176 mg, 0.5 mmol) and 2-hydroxy-propyl-beta-cyclodextrin (Biosynth Carbosynth, 1.46 g, 1 mmol) were volumetrically mixed in a mortar. Water was added to obtain a viscous paste using mortar and pestle. The paste was mixed for 5 minutes and dried over night at 60 degrees centigrade. A white powder comprising 10.8% (w/w) griseofulvin was isolated.

### Intermediate 13 Phenytoin 2-Hydroxypropyl-Beta-Cyclodextrin Complex (1:1.2)

Phenytoin (DDL, 252 mg, 1 mmol) and 2-hydroxy-propyl-beta-cyclodextrin (Biosynth Carbosynth, 1.752 g, 1.2 mmol) were volumetrically mixed in a mortar. Water was added to obtain a viscous paste using mortar and pestle. The paste was mixed for 5 minutes and dried over night at 60 degrees centigrade. A white powder comprising 12.6% (w/w) phenytoin was isolated.

### Intermediate 14 Phenobarbital 2-Hydroxypropyl-Beta-Cyclodextrin Complex (1:1.2)

Phenobarbital (DDL, 232 mg, 1 mmol) and 2-hydroxy-propyl-beta-cyclodextrin (Biosynth Carbosynth, 1.752 g, 1.2 mmol) were volumetrically mixed in a mortar. Water was added to obtain a viscous paste using mortar and pestle. The paste was mixed for 5 minutes and dried over night at 60 degrees centigrade. A white powder comprising 11.7% (w/w) phenobarbital was isolated.

### Intermediate 15 Phenytoin 2-Hydroxypropyl-Beta-Cyclodextrin Complex (1:1.2)

Phenytoin (DDL, 252 mg, 1 mmol) and 2-hydroxy-propyl-beta-cyclodextrin (Biosynth Carbosynth, 1.752 g, 1.2 mmol) were volumetrically mixed in a mortar. Absolute alcohol (3 ml) was added, the mixture was stirred for 5 minutes and dried over night at 60 degrees centigrade. A white powder comprising 12.6% (w/w) phenytoin was isolated.

### Intermediate 16 Amphotericin B Gamma-Cyclodextrin (1:1.2)

Amphotericin B (DDL, 924 mg, 1 mmol) and gamma-cyclodextrin (Cavamax W8, 1.556 g, 1.2 mmol) were volumetrically mixed in a mortar. Water was added to obtain a viscous paste using mortar and pestle. The paste was mixed for 5 minutes and dried over night at 60 degrees centigrade. A yellow powder comprising 37.3% (w/w) amphotericin B was isolated.

### Intermediate 17 Tetracycline Hydrochloride Methyl-Beta-Cyclodextrin Complex (1:1.5)

Tetracycline hydrochloride (DDL, 96 mg, 0.2 mmol) and methyl-beta-cyclodextrin (Aldrich, 396 mg 0.3 mmol) were volumetrically mixed in a mortar. Water was added to obtain a viscous paste using mortar and pestle. The paste was mixed for 5 minutes and dried over night at 60 degrees centigrade. A pale gray-green powder comprising 19.5% (w/w) tetracycline hydrochloride was isolated.

### Intermediate 18 Cytarabine Beta-Cyclodextrin Complex (1:1.5)

Cytarabine (DDL, 243 mg, 1 mmol) and beta-cyclodextrin (DDL, 2.003 g, 1.5 mmol) were volumetrically mixed in a mortar. Water was added to obtain a viscous paste using mortar and pestle. The paste was mixed for 5 minutes and dried over night at 60 degrees centigrade. A white powder comprising 10.8% (w/w) cytarabine was isolated.

### Intermediate 19 Amoxicillin Beta-Cyclodextrin Complex (1:1.5)

Amoxicillin trihydrate (DDL, 420 mg, 1 mmol) and beta-cyclodextrin (DDL, 2.003 g, 1.5 mmol) were volumetrically mixed in a mortar. Water was added to obtain a viscous paste using mortar and pestle. The paste was mixed for 5 minutes and dried over night at 60 degrees centigrade. A white powder comprising appr. 17% (w/w) amoxicillin was isolated.

### Intermediate 20 Phenytoin 4-Sulphobutyl-Beta-Cyclodextrin Complex (1:1.2)

Phenytoin (DDL, 232 mg, 1 mmol) and 4-sulphobutyl-beta-cyclodextrin (BiosynthCarbosynth, 2.69 gram, 1.2 mmol) were volumetrically mixed in a mortar. Water/ethanol (1:1 (v/v)) was added to obtain a viscous paste using mortar and pestle. The paste was mixed for 5 minutes and dried over night at 60 degrees centigrade. A white powder comprising 8.6% (w/w) phenytoin was isolated.

### Intermediate 21 Phenobarbital 4-Sulphobutyl-Beta-Cyclodextrin Complex (1:1.2)

Phenobarbital (DDL, 252 mg, 1 mmol) and 4-sulphobutyl-beta-cyclodextrin (BiosynthCarbosynth, 2.69 gram, 1.2 mmol) were volumetrically mixed in a mortar. Water/ethanol (1:1 (v/v)) was added to obtain a viscous paste using mortar and pestle. The paste was mixed for 5 minutes and dried over night at 60 degrees centigrade. A white powder comprising 7.9% (w/w) phenobarbital was isolated.

### Intermediate 22 Griseofulvin 4-Sulphobutyl-Beta-Cyclodextrin Complex (1:1.2)

Griseofulvin (Sigma Aldrich, 352 mg, 1 mmol) and 4-sulphobutyl-beta-cyclodextrin (BiosynthCarbosynth, 2.69 gram, 1.2 mmol) were volumetrically mixed in a mortar. Water/ethanol (1:1 (v/v)) was added to obtain a viscous paste using mortar and pestle. The paste was mixed for 5 minutes and dried over night at 60 degrees centigrade. A white powder comprising 11.6% (w/w) griseofulvin was isolated.

### Intermediate 23 Prednisolon 4-Sulphobutyl-Beta-Cyclodextrin Complex (1:1.2)

Prednisolon (Sigma Aldrich, 360 mg, 1 mmol) and 4-sulphobutyl-beta-cyclodextrin (BiosynthCarbosynth, 2.69 gram, 1.2 mmol) were volumetrically mixed in a mortar. Water/ethanol (1:1 (v/v)) was added to obtain a viscous paste using mortar and pestle. The paste was mixed for 5 minutes and dried over night at 60 degrees centigrade. A white powder comprising 11.8% (w/w) prednisolon was isolated.

### Intermediate 24 Rapamycin Beta-Cyclodextrin Complex (1:2)

Rapamycin (MedChem express, 100 mg) and 2-hydroxypropyl-beta-cyclodextrin (Aldrich, 320 mg) were volumetrically mixed with a pestle in a mortar for 5 minutes with addition of a few drops of water to obtain a viscous paste. The paste was dried under vacuum overnight at room temperature. A white powder comprising 23.8% (w/w) rapamycin was isolated.

### Intermediate 25 Preparation of Aggregated Amorphous cCVD-SP

Aggregated amorphous cCVD-SP like H18 particles were produced by CVD in a reactor where the reactor comprise a reactor body and a rotation device operatively arranged to the reactor, wherein the rotation device is configured to rotate the reactor around an axis during production according to WO2013048258. The process for preparation of stable aggregates like particle type H18 not free non-aggregated particles relates to control of process parameters as described below:

Silane starts to decompose at about 400° C. The process is a gradual process where silane decomposes and forms higher order silanes that in turn forms rings and stacks. When the higher order silanes starts stacking to 3d structures they are classified as a nuclei which will scavenge silanes and grow into larger particles. Depending on the growth rate these particles may grow faster than they release hydrogen and thus they will constitute both silicon, and silicon hydride where the gradient of hydrogen content is larger towards the surface. If the growth rate is high but the surface is kept cold the silicon hydride surface will be sticky and collisions between particles will lead to agglomeration. To intentionally form agglomerates it is thus important to keep the growth rate high, the hydrogen release slow, the number of particles pr volume high and have a process with substantially residence time to allow for many particle collisions before the process is stopped and the particles harvested.

### Example 1 Amorphous cCVD-SP Comprising Atorvastatin Calcium

Atorvastatin beta-cyclodextrin complex (intermediate 1, 500 mg) was dissolved in ethanol (10 ml). Silicon particles (batch RIF1, amorphous silicon average diameter 554 nm, PDI 0.164, 50 mg) were suspended in 1 ml of the ethanol solution comprising atorvastatin beta-cyclodextrin complex (intermediate 1) in a micro-centrifuge vial. The mixture was sonicated for 10 minutes in a sonicator bath at 70 degrees centigrade, centrifuge (14 000×, 8 minutes) and dried at 60 degrees centigrade until constant weight. The weight was 32 mg higher than reference sample (same particles treated by pure water). The product comprised appr. 39% atorvastatin beta-cyclodextrin complex

### Example 2 Amorphous cCVD-SP Comprising Metformin Hydrochloride

Metformin hydrochloride (Ph.Eur, Weifa), 1.5 g) was dissolved in water (10 ml). Silicon particles (Batch RIF1, amorphous silicon, average diameter 554 nm, PDI 0.164 50 mg) were suspended in 1 ml of the aqueous solution comprising metformin hydrochloride in a micro-centrifuge vial. The mixture was sonicated for 10 minutes in a sonicator bath at 70 degrees centigrade, centrifuge (14 000×, 8 minutes) and dried at 60 degrees centigrade until constant weight. The weight was 24 mg higher than reference sample (same particles treated by pure water). The product comprised appr. 32% metformin hydrochloride.

### Example 3 Amorphous cCVD-SP Comprising Metformin Losartan Potassium

Losartan potassium (DDL, 1.5 g) was dissolved in water (10 ml). Silicon particles (batch RIF1, amorphous silicon, average diameter 554 nm, PDI 0.164, 50 mg) were suspended in 1 ml of the aqueous solution comprising losartan potassium in a micro-centrifuge vial. The mixture was sonicated for 10 minutes in a sonicator bath at 70 degrees centigrade, centrifuge (14 000×, 8 minutes) and dried at 60 degrees centigrade until constant weight. The weight was 16 mg higher than reference sample (same particles treated by pure water). The product comprised appr. 24% losartan potassium.

### Example 4 Crystalline cCVD-SP Comprising Atorvastatin Calcium

Atorvastatin-2-hydroxypropyl beta-cyclodextrin complex (intermediate 1, 500 mg) was dissolved in ethanol (10 ml). Silicon particles (batch R4F1, crystalline silicon. average diameter 117 nm, PDI 0.277, 50 mg) were suspended in 1 ml of the ethanol solution comprising atorvastatin beta-cyclodextrin complex in a micro-centrifuge vial. The mixture was sonicated for 10 minutes in a sonicator bath at 70 degrees centigrade, centrifuge (14 000×, 8 minutes) and dried at 60 degrees centigrade until constant weight. The weight was 10 mg higher than reference sample (same particles treated by pure water). The product comprised appr. 17% atorvastatin beta-cyclodextrin complex

### Example 5 Crystalline cCVD-SP Comprising Metformin Hydrochloride

Metformin hydrochloride (Ph.Eur, Weifa), 1.5 g) was dissolved in water (10 ml). Silicon particles (batch R4F1, crystalline silicon, average diameter 117 nm, PDI 0.277, 50 mg) were suspended in 1 ml of the aqueous solution comprising metformin hydrochloride in a micro-centrifuge vial. The mixture was sonicated for 10 minutes in a sonicator bath at 70 degrees centigrade, centrifuge (14 000×, 8 minutes) and dried at 60 degrees centigrade until constant weight. The weight was 12 mg higher than reference sample (same particles treated by pure water). The product comprised appr. 19% metformin hydrochloride.

### Example 6 Amorphous cCVD-SP Comprising Losartan Potassium

Losartan potassium (DDL, 1.5 g) was dissolved in water (10 ml). Porous silicon particles (batch RIF1, amorphous silicon, average diameter 554 nm, PDI 0.164, 50 mg) were suspended in 1 ml of the aqueous solution comprising losartan potassium in a micro-centrifuge vial. The mixture was sonicated for 10 minutes in a sonicator bath at 70 degrees centigrade, centrifuge (14 000×, 8 minutes) and dried at 60 degrees centigrade until constant weight. The weight was 29 mg higher than reference sample (same particles treated by pure water). The product comprised 37% losartan potassium.

### Example 7 Amorphous cCVD-SP Aggregates Comprising Griseofulvin

Griseofulvin (DDL, 50 mg) was dissolved in dimethylformamide (DMF) (0.5 ml). The solution was dropped into amorphous silicon particles (450 mg) in a mortar. The particles were in the form of stable aggregates (batch no. H18, average aggregate diameter 210 nm, PDI 0.190). The mixture was stirred in the mortar with a pestle for 5 minutes forming a paste. The mortar with pestle was dried in a high vacuum oven at 50 degrees centigrade for 24 hours. The dry particles were scraped out of the mortar. The particles comprised of 10% w/w griseofulvin.

### Example 8 Amorphous cCVD-SP Comprising Griseofulvin

Griseofulvin (DDL, 50 mg) was dissolved in dimethylformamide (DMF) (0.5 ml). The solution was dropped into amorphous silicon particles (batch no. R8F2, 450 mg) in a mortar. The mixture was stirred in the mortar with a pestle for 5 minutes forming a paste. The mortar with pestle was dried in a high vacuum oven at 50 degrees centigrade for 24 hours. The dry particles were scraped out of the mortar. The particles comprised of 10% w/w griseofulvin.

### Example 9 Amorphous cCVD-SP Comprising Griseofulvin

Griseofulvin (DDL, 50 mg) was dissolved in dimethylformamide (DMF) (0.5 ml). The solution was dropped into amorphous silicon particles (batch no. F26F2, SEM size 200-400 nM, 450 mg) in a mortar. The mixture was stirred in the mortar with a pestle for 5 minutes forming a paste. The mortar with pestle was dried in a high vacuum oven at 50 degrees centigrade for 24 hours. The dry particles were scraped out of the mortar. The particles comprised of 10% w/w griseofulvin.

### Example 10 Amorphous cCVD-SP Comprising Erythromycin

Erythromycin (DDL, 100 mg) was dissolved in dimethylformamide (DMF) (1 ml). The solution was dropped into amorphous silicon particles (batch no. R8F2, 900 mg) in a mortar. The mixture was stirred in the mortar with a pestle for 5 minutes forming a paste. The mortar with pestle was dried in a high vacuum oven at 50 degrees centigrade for 24 hours. The dry particles were scraped out of the mortar. The particles comprised of 10% w/w erythromycin.

The release of erythromycin from the particles to water was studied over time using HPLC.

HPLC system: HP1100. Column: Zorbax Extend C-18, 4.6×250 mm, 5 um, mobile phase; 80% methanol and 20% 0.01M K2HPO4, flow: 1 ml/min, injection volume 10 ul, detection wave length. 286 nm, run time: 12 min.

The release of erythromycin from the particles at 2 hours was 290% compared to the release from free erythromycin powder.

### Example 11 Amorphous cCVD-SP Comprising Erythromycin

Erythromycin (100 mg) was dissolved in dimethylformamide (DMF) (1 ml). The solution was dropped into amorphous silicon particles (batch no. F26F2, SEM size 200-400 nM, 900 mg) in a mortar. The silicon particle size was 100-300 nm. The mixture was stirred in the mortar with a pestle for 5 minutes forming a paste. The mortar with pestle was dried in a high vacuum oven at 50 degrees centigrade for 24 hours. The dry particles were scraped out of the mortar. The particles comprised of 10% w/w erythromycin.

(The mortar and pestle was cleaned in 2 M sodium hydroxide and washed with water before preparation of a new batch silicon particles.)

The release of erythromycin from the particles to water was studied over time using HPLC.

HPLC system: HP1100. Column: Zorbax Extend C-18, 4.6×250 mm, 5 um, mobile phase; 80% methanol and 20% 0.01M K2HPO4, flow: 1 ml/min, injection volume 10 ul, detection wave length. 286 nm, run time: 12 min.

The release of erythromycin from the particles at 2 hours was 258% compared to the release from free erythromycin powder.

### Example 12 Amorphous cCVD-SP Comprising Erythromycin

Erythromycin (300 mg) was dissolved in dimethylformamide (DMF) (1.5 ml). The solution was dropped into amorphous silicon particles (batch no. F26F2, SEM size 200-400 nM, 900 mg) in a mortar. The silicon particle size was 100-300 nm. The mixture was added more DMF (3 ml) to secure good contact with the fluffy particles, stirred in the mortar with a pestle for 5 minutes forming a paste. The mortar with pestle was dried in a high vacuum oven at 50 degrees centigrade for 24 hours. The dry particles were scraped out of the mortar. The particles comprised of 10% w/w erythromycin.

The release of erythromycin from the particles to water was studied over time using HPLC.

HPLC system: HP1100. Column: Zorbax Extend C-18, 4.6×250 mm, 5 um, mobile phase; 80% methanol and 20% 0.01M K2HPO4, flow: 1 ml/min, injection volume 10 ul, detection wave length. 286 nm, run time: 12 min.

The release of erythromycin from the particles at 2 hours was 261% compared to the release from free erythromycin powder.

### Example 13 Amorphous cCVD-SP Aggregates Comprising Erythromycin

Erythromycin (50 mg) was dissolved in dimethylformamide (DMF) (0.5 ml). The solution was dropped into amorphous silicon particles (450 mg) in a mortar. The particles were in the form of stable aggregates (batch no. H18, average aggregate diameter 210 nm, PDI 0.190). The mixture was stirred in the mortar with a pestle for 5 minutes forming a paste. The mortar with pestle was dried in a high vacuum oven at 50 degrees centigrade for 24 hours. The dry particles were scraped out of the mortar. The particles comprised of 10% w/w erythromycin.

The release of erythromycin from the particles to water was studied over time using HPLC.

HPLC system: HP1100. Column: Zorbax Extend C-18, 4.6×250 mm, 5 um, mobile phase; 80% methanol and 20% 0.01M K2HPO4, flow: 1 ml/min, injection volume 10 ul, detection wave length. 286 nm, run time: 12 min.

The release of erythromycin from the particles at 2 hours was 219% compared to the release from free erythromycin powder.

### Example 14 Amorphous cCVD-SP Aggregates Comprising Griseofulvin-2-Hydroxypropyl-Beta-Cyclodextrin

Griseofulvin-2-hydroxypropyl-beta-cyclodextrin (Intermediate 2, 50 mg) was dissolved in dimethylformamide (DMF) (0.5 ml). The solution was dropped into amorphous silicon particles (450 mg) in a mortar. The particles were in the form of stable aggregates (batch no. H18. average aggregate diameter 210 nm, PDI 0.190). The mixture was stirred in the mortar with a pestle for 5 minutes forming a paste. The mortar with pestle was dried in a high vacuum oven at 50 degrees centigrade for 24 hours. The dry particles were scraped out of the mortar. The particles comprised of 0.74% w/w griseofulvin.

### Example 15 Amorphous cCVS-SP Aggregates Comprising Erythromycin-2-Hydroxypropyl-Beta-Cyclodextrin

Erythromycin-2-hydroxypropyl-beta-cyclodextrin (Intermediate 10, 50 mg) was dissolved in absolute ethanol (0.5 ml). The solution was dropped into amorphous silicon particles (450 mg) in a mortar. The particles were in the form of stable aggregates (batch no. H18, average aggregate diameter 210 nm, PDI 0.190). The mixture was stirred in the mortar with a pestle for 5 minutes forming a paste. The mortar with pestle was dried at 60 degrees centigrade for 24 hours. The dry particles were scraped out of the mortar. The particles comprised of 0.74% w/w erythromycin.

### Example 16 Amorphous cCVD-SP Aggregates Comprising Griseofulvin-2-Hydroxypropyl-Beta-Cyclodextrin

Griseofulvin-2-hydroxypropyl-beta-cyclodextrin (Intermediate 11, 50 mg) was dissolved in absolute ethanol (1 ml) by heating. The solution was dropped into amorphous silicon particles (450 mg) in a mortar. The particles were in the form of aggregates (batch no. H18 average aggregate diameter 210 nm, PDI 0.190). The mixture was stirred in the mortar with a pestle for 5 minutes forming a paste. The mortar with pestle was dried in a high vacuum oven at 50 degrees centigrade for 24 hours. The dry particles were scraped out of the mortar. The particles comprised of 1.94% w/w griseofulvin.

The release of griseofulvin from the particles to water was studied over time using HPLC.

HPLC system: HP1100. Column: PLRP-S, 2.1×50 mm, 3 um, mobile phase; 40% acetonitrile and 0.1% HCOOH, flow: 0.2 ml/min, injection volume 2.4 ul, detection wave length. 286 nm, run time: 8 min.

The release of griseofulvin from the particles at 2 hours was 99% compared to the release from free griseofulvin powder. The release of griseofulvin from the particles at 2 hours was 96% compared to the release from free griseofulvin-2-hydroxypropyl-beta-cyclodextrin powder (intermediate11).

### Example 17 Amorphous cCVD-SP Aggregates Comprising Griseofulvin-2-Hydroxypropyl-Beta-Cyclodextrin

Griseofulvin-2-hydroxypropyl-beta-cyclodextrin (Intermediate 11, 50 mg) was dissolved in dimethylformamide (0.5 ml). The solution was dropped into amorphous silicon particles (450 mg) in a mortar. The particles were in the form of stable aggregates (batch no. H18, average aggregate diameter 210 nm, PDI 0.190). The mixture was stirred in the mortar with a pestle for 5 minutes forming a paste. The mortar with pestle was dried in a high vacuum oven at 50 degrees centigrade for 24 hours. The dry particles were scraped out of the mortar. The particles comprised of 20% w/w griseofulvin-2-hydroxypropyl-beta-cyclodextrin.

The release of griseofulvin from the particles to water was studied over time using HPLC.

HPLC system: HP1100. Column: PLRP-S, 2.1×50 mm, 3 um, mobile phase; 40% acetonitrile and 0.1% HCOOH, flow: 0.2 ml/min, injection volume 2.4 ul, detection wave length. 286 nmnm, run time: 8 min.

The release of griseofulvin from the particles at 2 hours was 130% compared to the release from free griseofulvin powder. The release of griseofulvin from the particles at 2 hours was 125% compared to the release from free griseofulvin-2-hydroxypropyl-beta-cyclodextrin powder (intermediate11).

### Example 18 Amorphous cCVD-SP Aggregates Comprising Griseofulvin

Griseofulvin (SigmaAldrich, 50 mg) was dissolved in dimethylformamide (0.5 ml). The solution was dropped into amorphous silicon particles (450 mg) in a mortar. The particles were in the form of aggregates (batch no. H18 average aggregate diameter 210 nm, PDI 0.190). The mixture was stirred in the mortar with a pestle for 5 minutes forming a paste. The mortar with pestle was dried in a high vacuum oven at 50 degrees centigrade for 24 hours. The dry particles were scraped out of the mortar. The particles comprised of 10% w/w griseofulvin.

The release of griseofulvin from the particles to water was studied over time using HPLC.

HPLC system: HP1100. Column: PLRP-S, 2.1×50 mm, 3 um, mobile phase; 40% acetonitrile and 0.1% HCOOH, flow: 0.2 ml/min, injection volume 2.4 ul, detection wave length. 286 nmnm, run time: 8 min.

The release of griseofulvin from the particles at 2 hours was 109% compared to the release from free griseofulvin powder.

### Example 19 Amorphous cCVD-SP Aggregates Comprising Erythromycin-2-Hydroxypropyl-Beta-Cyclodextrin

Erythromycin-2-hydroxypropyl-beta-cyclodextrin (Intermediate 10, 100 mg) was dissolved in dimethylformamide (0.5 ml). The solution was dropped into amorphous silicon particles (400 mg) in a mortar. The particles were in the form of aggregates (batch no. H18, average aggregate diameter 210 nm, PDI 0.190). The mixture was stirred in the mortar with a pestle for 5 minutes forming a paste. The mortar with pestle was dried in a high vacuum oven at 50 degrees centigrade for 24 hours. The dry particles were scraped out of the mortar. The particles comprised of 6.7% w/w erythromycin.

The release of erythromycin from the particles to water was studied over time using HPLC.

HPLC system: HP1100. Column: Zorbax Extend C-18, 4.6×250 mm, 5 um, mobile phase; 80% methanol aND 20% 0.01M K2HPO4, flow: 1 ml/min, injection volume 10 ul, detection wave length. 286 nm, run time: 12 min.

The release of erythromycin from the particles at 2 hours was 291% compared to the release from free erythromycin powder. The release of erythromycin from the particles at 2 hours was 470% compared to the release from free erythromycin-2-hydroxypropyl-beta-cyclodextrin (intermediate 10).

### Example 20 Amorphous cCVD-SP Aggregates Comprising Cyclosporine and Additives

Cyclosporin together with pharmaceutical additives were extracted from capsules (4 Sandimmun Neooral 25 mg/Novartis)). The capsules were opened and extracted with absolute alcohol (5 ml). The alcohol was evaporated and the final solution was dissolved in absolute alcohol (2 ml) and was dropped into amorphous silicon particles (400 mg) in a mortar. The particles were in the form of aggregates (batch no. H18, average aggregate diameter 210 nm, PDI 0.190). The mixture was stirred in the mortar with a pestle for 5 minutes forming a paste. The mortar with pestle was dried at 60 degrees centigrade for 24 hours. The particulate material were scraped out of the mortar. The particulate material comprised of about 25% w/w cyclosporine including some additives from the Neooral formulation.

### Example 21 Amorphous cCVD-SP Aggregates Comprising Aciclovir-2-Hydroxypropyl-Beta-Cyclodextrin

Aciclovir-2-hydroxypropyl-beta-cyclodextrin (Intermediate 7, 100 mg) was dissolved in absolute ethanol (1.2 ml) by heating. The solution was dropped into amorphous silicon particles (400 mg) in a mortar. The particles were in the form of aggregates (batch no. H18, average aggregate diameter 210 nm, PDI 0.190). The mixture was stirred in the mortar with a pestle for 5 minutes forming a paste. The mortar with pestle was dried at 60 degrees centigrade for 24 hours. The dry particles were scraped out of the mortar. The particles comprised of 20% w/w aciclovir-2-hydroxypropyl-beta-cyclodextrin (1:1).

### Example 22 Amorphous cCVD-SP Aggregates Comprising Celecoxib

Celecoxib (DDL, 100 mg) was dissolved in absolute ethanol (0.7 ml). The solution was dropped into amorphous silicon particles (400 mg) in a mortar. The particles were in the form of aggregates (batch no. H18, average aggregate diameter 210 nm, PDI 0.190). The mixture was stirred in the mortar with a pestle for 5 minutes forming a paste. The mortar with pestle was dried at 60 degrees centigrade for 24 hours. The dry particles were scraped out of the mortar. The particles comprised of 20% celecoxib.

The release of celecoxib from the particles to water was studied over time using HPLC.

HPLC system: HP1100. Column: Zorbax Extend C-18, 4.6×250 mm, 5 um, mobile phase: 80% methanol and 20% 0.01M K2HPO4, flow: 1 ml/min, injection volume5 ul, detection wave length. 250 nm, run time: 7 min.

The release of celecoxib from the particles at 2 hours and 4 hours was 2.1 times higher than for free celecoxib powder.

The release of celecoxib from the particles at 6 hours was 2.0 times higher than for free celecoxib powder.

### Example 23 Amorphous cCVD-SP Aggregates Comprising Griseofulvin

Griseofulvin (SigmaAldrich, 200 mg) was dissolved in dimethylformamide (0.7 ml). The solution was dropped into amorphous silicon particles (200 mg) in a mortar. The particles were in the form of aggregates (batch no. H18, average aggregate diameter 210 nm, PDI 0.190). The mixture was stirred in the mortar with a pestle for 5 minutes forming a paste. The mortar with pestle was dried at high vacuum at 50 degrees centigrade for 24 hours. The dry particles were scraped out of the mortar. The particles comprised of 50% w/w griseofulvin.

The release of griseofulvin from the particles to water was studied over time using HPLC.

HPLC system: HP1100. Column: PLRP-S, 2.1×50 mm, 3 um, mobile phase; 40% acetonitrile and 0.1% HCOOH, flow: 0.2 ml/min, injection volume 2.4 ul, detection wave length. 286 nm, run time: 8 min.

The release of griseofulvin from the particles at 2 hours was 70% compared to the release from free griseofulvin powder.

### Example 24 Amorphous cCVD-SP Aggregates Comprising Atorvastatin-2-Hydroxypropyl-Beta-Cyclodextrin

Atorvastatin-2-hydroxypropyl-beta-cyclodextrin (Intermediate 6, 100 mg) was dissolved in dimethylformamide (0.7 ml) by heating. The solution was dropped into amorphous silicon particles (400 mg) in a mortar. The particles were in the form of aggregates (batch no. H18 average aggregate diameter 210 nm, PDI 0.190). The mixture was stirred in the mortar with a pestle for 5 minutes forming a paste. The mortar with pestle was dried at high vacuum at 50 degrees centigrade for 24 hours. The dry particles were scraped out of the mortar. The particles comprised of 20% w/w atorvastatin calcium-2-hydroxypropyl-beta-cyclodextrin (1:1).

The release of atorvastatin from the particles to water was studied over time using HPLC.

HPLC system: HP1100. Column: PLRP-S, 2.1×50 mm, 3 um, mobile phase; 40% acetonitrile and 0.1% HCOOH, flow: 0.2 ml/min, injection volume 2.4 ul, detection wave length. 240 nm, run time: 3 min.

The release of atorvastatin from the particles at 2 hours was 140% compared to the release from free atorvastatin powder. The release of atorvastatin from the particles at 2 hours was 140% compared to the release from free atorvastatin-2-hydroxypropyl-beta-cyclodextrin (intermediate 6).

### Example 25 Amorphous cCVD-SP Aggregates Comprising Nystatin-2-Hydroxypropyl-Beta-Cyclodextrin

Nystatin-2-hydroxypropyl-beta-cyclodextrin (Intermediate 8, 100 mg) was dissolved in dimethylformamide (0.6 ml) by heating. The solution was dropped into amorphous silicon particles (400 mg) in a mortar. The particles were in the form of aggregates (batch no. H18 average aggregate diameter 210 nm, PDI 0.190). The mixture was stirred in the mortar with a pestle for 5 minutes forming a paste. The mortar with pestle was dried at high vacuum at 50 degrees centigrade for 24 hours. The dry particles were scraped out of the mortar. The particles comprised of 20% w/w nystatin-2-hydroxypropyl-beta-cyclodextrin (1:1).

### Example 26 Amorphous cCVD-SP Aggregates Comprising Losartan-2-Hydroxypropyl-Beta-Cyclodextrin

Losartan-2-hydroxypropyl-beta-cyclodextrin (Intermediate 5, 100 mg) was dissolved in dimethylformamide (0.7 ml) by heating. The solution was dropped into amorphous silicon particles (400 mg) in a mortar. The particles were in the form of aggregates (batch no. H18, average aggregate diameter 210 nm, PDI 0.190). The mixture was stirred in the mortar with a pestle for 5 minutes forming a paste. The mortar with pestle was dried at high vacuum at 50 degrees centigrade for 24 hours. The dry particles were scraped out of the mortar. The particles comprised of 20% w/w losartan potassium-2-hydroxypropyl-beta-cyclodextrin (1:3).

The release of losartan from the particles to water was studied over time using HPLC.

HPLC system: HP1100. Column: PLRP-S, 2.1×50 mm, 3 um, mobile phase; 40% acetonitrile and 0.1% HCOOH, flow: 0.2 ml/min, injection volume 2.4 ul, detection wave length. 226 nm. run time: 5 min.

The release of losartan from the particles at 2 hours was 56% compared to the release from free losartan potassium powder. The release of losartan from the particles at 2 hours was 70% compared to the release from free losartan-2-hydroxypropyl-beta-cyclodextrin (intermediate 5) powder.

### Example 27 Amorphous cCVD-SP Aggregates Comprising Aciclovir

Aciclovir (DDL, 100 mg) was dissolved in dimethylsulfoxide (0.5 ml) by heating. The solution was dropped into amorphous silicon particles (400 mg) in a mortar. The particles were in the form of aggregates (batch no. H18, average aggregate diameter 210 nm, PDI 0.190). The mixture was stirred in the mortar with a pestle for 5 minutes forming a paste. The mortar with pestle was dried at high vacuum at 50 degrees centigrade for 24 hours. The dry particles were scraped out of the mortar. The particles comprised of 20% w/w aciclovir.

### Example 28 Amorphous cCVD-SP Aggregates Comprising Chloramphenicol

Chloramphenicol (SigmaAldrich, 100 mg) was dissolved in dimethylsulfoxide (0.5 ml) by heating. The solution was dropped into amorphous silicon particles (400 mg) in a mortar. The particles were in the form of aggregates (batch no. H18, average aggregate diameter 210 nm, PDI 0.190). The mixture was stirred in the mortar with a pestle for 5 minutes forming a paste.

The mortar with pestle was dried at high vacuum at 50 degrees centigrade for 24 hours. The dry particles were scraped out of the mortar. The particles comprised of 20% w/w chloramphenicol.

The release of chloramphenicol from the particles to water was studied over time using HPLC.

HPLC system: HP1100. Column: PLRP-S, 2.1×50 mm, 3 um, mobile phase; 40% acetonitrile and 0.1% HCOOH, flow: 0.2 ml/min, injection volume 2.4 ul, detection wave length. 278 nm, run time: 12 min.

The release of chloramphenicol from the particles at 2 hours was 63% compared to the release from free chloramphenicol powder. The release of chloramphenicol from the particles at 4 hours was 59% compared to the release from free chloramphenicol powder. The release of chloramphenicol from the particles at 5 hours was 57% compared to the release from free chloramphenicol powder.

### Example 29 Crystalline cCVD-SP Comprising Chloramphenicol

Chloramphenicol (SigmaAldrich, 200 mg) was dissolved in dimethylsulfoxide (0.7 ml) by heating. The solution was dropped into crystalline silicon particles (800 mg, batch no. R5F3, average particle diameter 2332 nM, PDI 0.407) in a mortar. The mixture was stirred in the mortar with a pestle for 5 minutes forming a paste. The mortar with pestle was dried at high vacuum at 50 degrees centigrade for 24 hours. The dry particles were scraped out of the mortar. The particles comprised of 20% w/w chloramphenicol.

The release of chloramphenicol from the particles to water was studied over time using HPLC.

HPLC system: HP1100. Column: PLRP-S, 2.1×50 mm, 3 um, mobile phase; 40% acetonitrile and 0.1% HCOOH, flow: 0.2 ml/min, injection volume 2.4 ul, detection wave length. 278 nm, run time: 12 min.

The release of chloramphenicol from the particles at 2 hours was 85% compared to the release from free chloramphenicol powder. The release of chloramphenicol from the particles at 4 hours was 98% compared to the release from free chloramphenicol powder. The release of chloramphenicol from the particles at 5 hours was 100% compared to the release from free chloramphenicol powder.

### Example 30 Crystalline cCVD-SP Comprising Prednisolon

Prednisolon (SigmaAldrich, 200 mg) was dissolved in dimethylformamide (1.0 ml) by heating. The solution was dropped into crystalline silicon particles (800 mg, batch no. R5F3, average particle diameter 2332 nm, PDI 0.407) in a mortar. The mixture was stirred in the mortar with a pestle for 5 minutes forming a paste. The mortar with pestle was dried at high vacuum at 50 degrees centigrade for 24 hours. The dry particles were scraped out of the mortar. The particles comprised of 20% w/w prednisolon.

### Example 31 Crystalline cCVD-SP Comprising Aciclovir

Aciclovir (DDL, 200 mg) was dissolved in dimethylsulphoxide (1.0 ml) by heating. The solution was dropped into crystalline silicon particles (800 mg, batch no. R5F3, average particle diameter 2332 nm, PDI 0.407) in a mortar. Particle size was 100-300 nm. The mixture was stirred in the mortar with a pestle for 5 minutes forming a paste. The mortar with pestle was dried at high vacuum at 50 degrees centigrade for 24 hours. The dry particles were scraped out of the mortar. The particles comprised of 20% w/w aciclovir.

### Example 32 Amorphous cCVD-SP Aggregates Comprising Phenytoin

Phenytoin (DDL, 100 mg) was dissolved in dimethylformamide (0.5 ml) by heating. The solution was dropped into amorphous silicon particles (900 mg) in a mortar. The particles were in the form of stable amorphous aggregates (batch no. H18 average aggregate diameter 210 nm, PDI 0.190). The mixture was stirred in the mortar with a pestle for 5 minutes forming a paste. The mortar with pestle was dried at high vacuum at 50 degrees centigrade for 24 hours. The dry particles were scraped out of the mortar. The particles comprised of 10% w/w phenytoin.

The release of phenytoin from the particles to water was studied over time using HPLC.

HPLC system: HP1100. Column: Zorbax Extend C-18, 4.6×250 mm, 5 um, mobile phase; 50% acetonitrile, flow: 1 ml/min, injection 5 ul, detection wave length. 210 and 200 nm, run time: 6 min.

The release of phenytoin from the particles at 2 hours was 134% compared to the release from free phenytoin powder.

### Example 33 Amorphous cCVD-SP Aggregates Comprising Phenobarbital

Phenobarbital (DDL, 100 mg) was dissolved in dimethylformamide (0.7 ml) by heating. The solution was dropped into amorphous silicon particles (900 mg) in a mortar. The particles were in the form of stable aggregates (batch no. H18, average aggregate diameter 210 nm, PDI 0.190). The mixture was stirred in the mortar with a pestle for 5 minutes forming a paste. The mortar with pestle was dried at high vacuum at 50 degrees centigrade for 24 hours. The dry particles were scraped out of the mortar. The particles comprised of 10% w/w phenobarbital.

HPLC system: HP1100. Column: Zorbax Extend C-18, 4.6×250 mm, 5 um, mobile phase; 50% acetonitrile, flow: 1 ml/min, injection 5 ul, detection wave length. 210 and 200 nm, run time: 6 min.

The release of phenobarbital from the particles at 2 hours was 174% compared to the release from free phenobarbital powder.

### Example 34 Amorphous cCVD-SP Aggregates Comprising Phenytoin 2-Hydroxypropyl-Beta-Cyclodextrin Complex

Phenytoin 2-hydroxypropyl-beta-cyclodextrin complex (Intermediate 15,100 mg) was dissolved in dimethylformamide (0.6 ml) by heating. The solution was dropped into amorphous silicon particles (900 mg) in a mortar. The particles were in the form of stable aggregates (batch no. H18, average aggregate diameter 210 nm, PDI 0.190). The mixture was stirred in the mortar with a pestle for 5 minutes forming a paste. The mortar with pestle was dried at high vacuum at 50 degrees centigrade for 24 hours. The dry particles were scraped out of the mortar. The particles comprised of 2.5% w/w phenytoin.

HPLC system: HP1100. Column: Zorbax Extend C-18, 4.6×250 mm, 5 um, mobile phase; 50% acetonitrile, flow: 1 ml/min, injection 5 ul, detection wave length. 210 and 200 nm, run time: 6 min.

The release of phenytoin from the particles at 2 hours was 206% compared to the release from free phenytoin powder. The release of phenytoin from the particles at 2 hours was 100% compared to the release from free 2-hydroxypropyl-beta-cyclodextrin complex (intermediate 15).

### Example 35 Amorphous cCVD-SP Aggregates Comprising Phenobarbital 2-Hydroxypropyl-Beta-Cyclodextrin Complex

Phenobarbital 2-hydroxypropyl-beta-cyclodextrin complex (Intermediate 14,100 mg) was dissolved in dimethylformamide (0.6 ml) by heating. The solution was dropped into amorphous silicon particles (900 mg) in a mortar. The particles were in the form of aggregates (batch no. H18, average aggregate diameter 210 nm, PDI 0.190) The mixture was stirred in the mortar with a pestle for 5 minutes forming a paste. The mortar with pestle was dried at high vacuum at 50 degrees centigrade for 24 hours. The dry particles were scraped out of the mortar. The particles comprised of 2.3% w/w phenobarbital.

HPLC system: HP1100. Column: Zorbax Extend C-18, 4.6×250 mm, 5 um, mobile phase; 50% acetonitrile, flow: 1 ml/min, injection 5 ul, detection wave length. 210 and 200 nm, run time: 6 min.

The release of phenobarbital from the particles at 2 hours was 290% compared to the release from free phenobarbital powder. The release of phenobarbital from the particles at 2 hours was 80% compared to the release from free phenobarbital 2-hydroxypropyl-beta-cyclodextrin complex (intermediate 14).

### Example 36 Amorphous cCVD-SP Aggregates Comprising Amphotericin B Gamma-Cyclodextrin

Amphotericin B-gamma-cyclodextrin complex (Intermediate 16,100 mg) was dissolved in dimethylformamide (0.6 ml) by heating. The solution was dropped into amorphous silicon particles (400 mg) in a mortar. The particles were in the form of stable aggregates (batch no. H18, average aggregate diameter 210 nm, PDI 0.190). The mixture was stirred in the mortar with a pestle for 5 minutes forming a paste. The mortar with pestle was dried at high vacuum at 50 degrees centigrade for 24 hours. The dry particles were scraped out of the mortar. The particles comprised of 7.5% amphotericin B.

### Example 37 Amorphous cCVD-SP Aggregates Comprising Tetracycline Hydrochloride Methyl-Beta-Cyclodextrin Complex

Tetracycline-HCl-methyl-beta-cyclodextrin complex (Intermediate 17,100 mg) was dissolved in dimethylformamide (1.0 ml) by heating. The solution was dropped into amorphous silicon particles (400 mg) in a mortar. The particles were in the form of stable aggregates (batch no. H18, average aggregate diameter 210 nm, PDI 0.190). The mixture was stirred in the mortar with a pestle for 5 minutes forming a paste. The mortar with pestle was dried at high vacuum at 50 degrees centigrade for 24 hours. The dry particles were scraped out of the mortar. The particles comprised of 3.9% tetracycline HCl.

### Example 38 Amorphous cCVD-SP Comprising Cytarabine Beta-Cyclodextrin Complex

Cytarabine beta-cyclodextrin complex (Intermediate 18,100 mg) was dissolved in dimethylformamide (0.5 ml) by heating. The solution was dropped into amorphous silicon particles (400 mg) in a mortar. The particles were in the form of stable aggregates (batch no. H18, average aggregate diameter 210 nm, PDI 0.190). The mixture was stirred in the mortar with a pestle for 5 minutes forming a paste. The mortar with pestle was dried at high vacuum at 50 degrees centigrade for 24 hours. The dry particles were scraped out of the mortar. The particles comprised of 2.2% (w/w) cytarabine.

### Example 39 Amorphous cCVD-SP Comprising Amoxicillin Beta-Cyclodextrin Complex

Amoxicillin beta-cyclodextrin complex (Intermediate 19,100 mg) was dissolved in dimethylformamide (0.5 ml) by heating. The solution was dropped into amorphous silicon particles (400 mg) in a mortar. The particles were in the form of stable aggregates (batch no. H18 average aggregate diameter 210 nm, PDI 0.190). The mixture was stirred in the mortar with a pestle for 5 minutes forming a paste. The mortar with pestle was dried at high vacuum at 50 degrees centigrade for 24 hours. The dry particles were scraped out of the mortar. The particles comprised of 3.4% (w/w) amoxicillin.

### Example 40 Amorphous cCVD-SP Aggregates Comprising Phenytoin 4-Sulphobutyl-Beta-Cyclodextrin Complex

Phenytoin 4-sulphobutyl-beta-cyclodextrin complex (Intermediate 20,100 mg) was dissolved in dimethylsulphoxide (0.5 ml) by heating. The solution was dropped into amorphous silicon particles (400 mg) in a mortar. The particles were in the form of stable aggregates (batch no. H18, average aggregate diameter 210 nm, PDI 0.190). The mixture was stirred in the mortar with a pestle for 5 minutes forming a paste. The mortar with pestle was dried at high vacuum at 50 degrees centigrade for 24 hours. The dry particles were scraped out of the mortar. The particles comprised of 1.7% (w/w) phenytoin.

### Example 41 Amorphous cCVD-SP Aggregates Comprising Phenobarbital 4-Sulphobutyl-Beta-Cyclodextrin Complex

Phenobarbital 4-sulphobutyl-beta-cyclodextrin complex (Intermediate 21,100 mg) was dissolved in dimethylsulphoxide (0.5 ml) by heating. The solution was dropped into amorphous silicon particles (400 mg) in a mortar. The particles were in the form of stable aggregates (batch no. H18, average aggregate diameter 210 nm, PDI 0.190). The mixture was stirred in the mortar with a pestle for 5 minutes forming a paste. The mortar with pestle was dried at high vacuum at 50 degrees centigrade for 24 hours. The dry particles were scraped out of the mortar. The particles comprised of 1.6% phenobarbital.

### Example 42 Amorphous cCVD-SP Comprising Griseofulvin 4-Sulphobutyl-Beta-Cyclodextrin Complex

Griseofulvin 4-sulphobutyl-beta-cyclodextrin complex (Intermediate 22,100 mg) was dissolved in dimethylsulphoxide (0.5 ml) by heating. The solution was dropped into amorphous silicon particles (400 mg) in a mortar. The particles were in the form of aggregates (batch no. H18, average aggregate diameter 210 nm, PDI 0.190). The mixture was stirred in the mortar with a pestle for 5 minutes forming a paste. The mortar with pestle was dried at high vacuum at 50 degrees centigrade for 24 hours. The dry particles were scraped out of the mortar. The particles comprised of 2.3% griseofulvin.

### Example 43 Amorphous cCVD-SP Comprising Prednisolon 4-Sulphobutyl-Beta-Cyclodextrin Complex

Prednisolon-4-sulphobutyl-beta-cyclodextrin complex (Intermediate 23,100 mg) was dissolved in dimethylformamide (0.5 ml) by heating. The solution was dropped into amorphous silicon particles (400 mg) in a mortar. The particles were in the form of aggregates (batch no. H18, average aggregate diameter 210 nm, PDI 0.190). The mixture was stirred in the mortar with a pestle for 5 minutes forming a paste. The mortar with pestle was dried at high vacuum at 50 degrees centigrade for 24 hours. The dry particles were scraped out of the mortar. The particles comprised of 2.3% prednisolon.

### Example 44 Polysorbate 80 Coated Amorphous cCVD-SP Aggregates Comprising Amphotericin B Gamma-Cyclodextrin

Amorphous cCVD-SP aggregates comprising amphotericin B gamma-cyclodextrin (from example 36, 50 mg) was suspended in an aqueous solution of Polysorbate 80 (DDL, 0.2% w/w, 1 ml). The mixture was sonicated for 5 minutes and centrifugated (14 000 rpm) for 5 minutes. The supernatant was removed and the particles were dried for 12 hours at 50 degrees centigrade.

### Example 45 Polysorbate 20 Coated Amorphous cCVD-SP Aggregates Comprising Tetracycline Hydrochloride Methyl-Beta-Cyclodextrin

Amorphous cCVD-SP aggregates comprising tetracycline hydrochloride methyl-beta-cyclodextrin (from example 37, 50 mg) was suspended in an aqueous solution of Polysorbate 20 (DDL, 0.2% w/w, 1 ml). The mixture was sonicated for 5 minutes and centrifugated (14 000 rpm) for 5 minutes. The supernatant was removed and the particles were dried for 12 hours at 50 degrees centigrade.

### Example 46 Cremophor EL Coated Amorphous cCVD-SP Aggregates Comprising Cytarabine Beta-Cyclodextrin

Amorphous cCVD-SP aggregates comprising cytarabine beta-cyclodextrin (from example 38, 50 mg) was suspended in an aqueous solution of Cremophor EL (Sigma, 0.2% w/w, 1 ml). The mixture was sonicated for 5 minutes and centrifugated (14 000 rpm) for 5 minutes. The supernatant was removed and the particles were dried for 12 hours at 50 degrees centigrade.

### Example 47 Amorphous cCVD-SP Comprising Rapamycin (50% Weight Load)

Preparation of rapamycin loaded particles: amorphous aggregated silicon particles with hydrodynamic size of 210 nm and PDI of 0.190 (batch no. H18) were first coated (adsorption, non-covalent coating) with Pluronic F-127 (Sigma). A 0.5% (w/v) solution of Pluronic F-127 was added 400 mg of H18 and subsequently treated with ultrasound in an ultrasonicator bath for 15 minutes, centrifuged, washed three times with water and vacuum dried over night after removal of the supernatant. Rapamycin (MedChem express) and Pluronic-coated H18 was weighed out and dissolved in dimethylformamide. The dispersion was treated with ultrasound in an ultrasonicator bath for 10 minutes before pipetting into aliquots containing 125 μg rapamycin each. The aliquots were dried under vacuum overnight.

Total weight of rapamycin: 5 mg

Total weight of Pluronic-coated H18: 5 mg

The particle product had a hydrodynamic size of 190.3 nm and a PDI of 0.362.

Release studies: one aliquot vial containing 0.125 mg rapamycin in Si particles, as prepared above, was chosen for release studies. The dried particle pellet was crushed into fine powder with a spatula. 1 ml of purified water was added and the suspension was ultrasonicated for 1 minute before adding to a round bottle with 50 ml buffer solution. The suspension was stirred for 48 hours at 37 degrees centigrade. Samples of 1 ml were withdrawn after 0.5, 1, 3, 6, 24, 30 and 48 hours, centrifuged for 6 minutes at 14 000 rpm and the supernatant was dried under vacuum (Speedvac concentrator) over night. Reconstitution of the sample with 500 μl methanol followed by centrifugation for removal of the salts (6 min, 14 000 rpm) was done before High-Performance Liquid Chromatography (HPLC, Surveyor Finnigan, Thermo) analysis. Quantification was done by comparing area under the chromatographic rapamycin peak to a chromatography standard curve made from rapamycin in methanol solutions with known concentrations. HPLC conditions: PLRP-S reversed phase column (1×150 mm, Agilent Technologies) set to 55 degrees centigrade, isocratic elution with mobile phase of 70% acetonitrile with 0.1% formic acid and 30% purified water with 0.1% formic acid, flow rate of 100 μl/min, injection volume of 20 μl and UV PDA detection. 278 nm was chosen as the peak absorption of rapamycin for quantification.

Buffer solution: Phosphate-buffered saline (PBS) at pH 7.4.

The release experiment was conducted 3 times. The average rapamycin release was plotted against time (FIG. 4). Control experiment: supersaturated solution of free rapamycin powder in PBS pH 7.4 and 37 degrees centigrade (2 experiments).

### Example 48 Amorphous cCVD-SP Comprising Rapamycin (10% Weight Load)

Preparation of rapamycin loaded particles was done as described in Example 47, with a rapamycin weight of 5 mg and a Pluronic-coated H18 weight of 45 mg used to obtain a 10% weight load. The particle product had a hydrodynamic size of 177.7 nm and a PDI of 0.147.

Release studies were done as described in Example 47 with PBS of pH 7.4 and PBS of pH 5.8, separately. 3 release experiments were conducted with each buffer solution. The average rapamycin release was plotted against time (FIG. 5).

### Example 49 Amorphous cCVD-SP Comprising Rapamycin (5% Weight Load)

Preparation of rapamycin loaded particles was done as described in Example 47, with a rapamycin weight of 4 mg and a Pluronic-coated H18 weight of 71 used to obtain a 5% weight load. The particle product had a hydrodynamic size of 154.5 nm and a PDI of 0.143.

Release studies were done as described in Example 47 with PBS of pH 7.4 and PBS of pH 5.8, separately. 3 release experiments were conducted with each buffer solution. The average rapamycin release was plotted against time (FIG. 6).

### Example 50 Amorphous cCVD-SP Comprising Rapamycin Beta-Cyclodextrin Complex (5% Weight Load Rapamycin)

Preparation of rapamycin loaded particles was done as described in Example 47, with a rapamycin-cyclodextrin complex (intermediate 24) weight of 16 mg and a Pluronic-coated H18 weight of 60 mg used to obtain a 5% rapamycin weight load. The particle product had a hydrodynamic size of 165.7 nm and a PDI of 0.141.

Release studies were done with PBS of pH 7.4 as described in Example 47. 2 release experiments were conducted. The average rapamycin release was plotted against time (FIG. 7). Control: rapamycin-cyclodextrin complex (intermediate 2).

### Example 51 Amorphous cCVD-SP Comprising Rapamycin Beta-Cyclodextrin Complex (10% Weight Load Rapamycin)

Preparation of rapamycin loaded particles was done as described in Example 47, with a rapamycin-cyclodextrin complex (intermediate 24) weight of 16 mg and a Pluronic-coated H18 weight of 22 mg used to obtain a 10% rapamycin weight load. The particle product had a hydrodynamic size of 168.3 and a PDI of 0.128.

Release studies were done with PBS of pH 7.4 as described in Example 47. 2 release experiments were conducted. The average rapamycin release was plotted against time (FIG. 7). Control: rapamycin-cyclodextrin complex (intermediate 24).

### Example 52 Accelerated Stability Studies of Amorphous cCVD-SP Comprising Rapamycin (10% Weight Load)

Three vials containing particle samples prepared as in Example 48 (10% weight load of rapamycin in Pluronic-coated H18 particles) were used for product stability studies. The vials were placed with a closed cap in a desiccator filled at the bottom with a saturated salt solution (NaCl) placed in a heat cabinet at 40 degrees centigrade, creating an atmosphere of 75% relative humidity (RH). These conditions represent accelerated stability studies of drug products and are inspired by the ICH guidelines Q1A. One vial was used for zero-point measurements, one vial was analyzed after 1 month and the third vial was analyzed after 2 months. Upon analysis was the particle pellet crushed with a spatula, 1 ml of methanol was added and the vial was ultrasound treated in an ultrasonicator bath for 15 minutes. The loaded rapamycin was thus extracted from the particles. The dispersion was left on the bench for a few hours before it was centrifuged (6 min, 14k rpm), the supernatant was withdrawn for rapamycin quantification by HPLC analysis. The particle pellet was saved for measurement of hydrodynamic size by DLS.

Hydrodynamic size of the particle batch changed little from 177.7 nm at 0 months, to 163.1 nm after 1 month and 173.8 nm after 2 months of storage under 40 degrees centigrade and 75% RH. The PDI value also changed little from 0.147 at 0 months, to 0.126 at 1 month and 0.169 at 2 months.

HPLC analysis for identification of rapamycin at time zero and after 1 month was conducted with a Zorbax C18 column (1×150 mm, 3.5 μm, Agilent), isocratic elution of 80% methanol with 0.1% trifluoroacetic acid and injection volume of 5 μl (other conditions as described in Example 47). HPLC analysis of the sample after 2 months storage was done as for the release sample analyses described in Example 47.

The rapamycin peak is seen after 2.6-2.9 min (FIG. 8). No occurrence of new peaks in the chromatogram indicates little degradation of rapamycin upon storage of the particle product under 40 degrees centigrade and 75% RH after 1 month and 2 months. This indicates stability of the drug product during storage at refrigerated conditions.

### Example 53 Amorphous cCVD Particles for Dual Delivery (Hydrogen Plus Erythromycin)

Particles prepared as in Example 13 (29 mg, NM014) were suspended in TRIS buffer (25 ml, pH 8.0) in a round bottle equipped with a tubing with a needle for hydrogen outlet in an inverted metered vial comprising water. The inverted vial is placed in a water bath (standard laboratory upset for collection of gas). The suspension was stirred at 37 degrees centigrade.

After 2 hours was 4 ml of hydrogen gas generated (equivalent to 154 ml/g Si). From example 13 was 219% of erythromycin released from the particles, comparing to free erythromycin powder, during 2 hours. After 21 hours was 7 ml hydrogen gas generated (equivalent to 269 ml/g Si).

### Example 54 Amorphous cCVD Particles for Dual Delivery (Hydrogen Plus Erythromycin)

Particles prepared as in Example 15 (31 mg, NM022) were tested for hydrogen generation as in Example 53. After 2 hours was 8 ml of hydrogen gas generated (equivalent to 286 ml/g Si). After 21 hours was 23 ml hydrogen gas generated (equivalent to 821 ml/g Si).

### Example 55 Amorphous cCVD Particles for Dual Delivery (Hydrogen Plus Griseofulvin)

Particles prepared as in Example 18 (51 mg, NM025) were tested for hydrogen generation as in Example 53. From example 18 was 109% of griseofulvin released from the particles, comparing to free griseofulvin powder, during 2 hours. After 21 hours was 22 ml hydrogen gas generated (equivalent to 478 ml/g Si).

### Example 56 Amorphous cCVD Particles for Dual Delivery (Hydrogen Plus Griseofulvin)

Particles prepared as in Example 16 (52 mg, NM023) were tested for hydrogen generation as in Example 53. After 2 hours was 13 ml of hydrogen gas generated (equivalent to 277 ml/g Si). From example 16 was 99% of griseofulvin released from the particles, comparing to free griseofulvin powder, during 2 hours. After 21 hours was 47 ml hydrogen gas generated (equivalent to 1000 ml/g Si).

### Example 57 Amorphous cCVD Particles for Dual Delivery (Hydrogen Plus Celecoxib)

Particles prepared as in Example 22 (52 mg, NM029) were tested for hydrogen generation as in Example 53. After 2 hours was 7.5 ml of hydrogen gas generated (equivalent to 179 ml/g Si). From example 22 was 210% of celecoxib released from the particles, comparing to free celecoxib powder, during 2 hours. After 4 hours was 12 ml hydrogen gas generated (equivalent to 286 ml/g Si).

### Example 58 Amorphous cCVD Particles for Dual Delivery (Hydrogen Plus Phenytoin)

Particles prepared as in Example 32 (57 mg, NM041) were tested for hydrogen generation as in Example 53. After 2 hours was 13 ml of hydrogen gas generated (equivalent to 253 ml/g Si). From example 32 was 134% of phenytoin released from the particles, comparing to free phenytoin powder, during 2 hours. After 16 hours was 34 ml hydrogen gas generated (equivalent to 663 ml/g Si).

### Example 59 Amorphous cCVD Particles for Dual Delivery (Hydrogen Plus Phenobarbital)

Particles prepared as in Example 33 (54 mg, NM042) were tested for hydrogen generation as in Example 53. After 2 hours was 40 ml of hydrogen gas generated (equivalent to 823 ml/g Si). From example 33 was 174% of phenobarbital released from the particles, comparing to free phenobarbital powder, during 2 hours. After 16 hours was 53 ml hydrogen gas generated (equivalent to 1090 ml/g Si).

### Example 60 Amorphous cCVD Particles for Dual Delivery (Hydrogen Plus Phenytoin)

Particles prepared as in Example 34 (63 mg, NM044) were tested for hydrogen generation as in Example 53. After 2 hours was 5 ml of hydrogen gas generated (equivalent to 99 ml/g Si). From example 34 was 206% of phenytoin released from the particles, comparing to free phenytoin powder, during 2 hours. After 16 hours was 40 ml hydrogen gas generated (equivalent to 794 ml/g Si).

### Example 61 Amorphous cCVD Particles for Dual Delivery (Hydrogen Plus Phenobarbital)

Particles prepared as in Example 35 (64 mg, NM045) were tested for hydrogen generation as in Example 53. After 2 hours was 7 ml of hydrogen gas generated (equivalent to 137 ml/g Si). From example 35 was 290% of phenobarbital released from the particles, comparing to free phenobarbital powder, during 2 hours. After 16 hours was 35 ml hydrogen gas generated (equivalent to 684 ml/g Si).

### Example 62 Amorphous cCVD Particles for Dual Delivery (Hydrogen Plus Griseofulvin)

Particles prepared as in Example 17 (63 mg, NM024) were tested for hydrogen generation as in Example 53. After 2 hours was 7.5 ml of hydrogen gas generated (equivalent to 149 ml/g Si). From example 17 was 130% of griseofulvin released from the particles, comparing to free griseofulvin powder, during 2 hours. After 3 days was 15 ml hydrogen gas generated (equivalent to 298 ml/g Si).

### Example 63 Amorphous cCVD Particles for Dual Delivery (Hydrogen Plus Griseofulvin)

Particles prepared as in Example 23 (81 mg, NM030) were tested for hydrogen generation as in Example 53. After 2 hours was 3 ml of hydrogen gas generated (equivalent to 74 ml/g Si). From example 23 was 70% of griseofulvin released from the particles, comparing to free griseofulvin powder, during 2 hours. After 16 hours was 22 ml hydrogen gas generated (equivalent to 544 ml/g Si).

### Example 64 Amorphous cCVD Particles for Dual Delivery (Hydrogen Plus Losartan)

Particles prepared as in Example 26 (57 mg, NM033) were tested for hydrogen generation as in Example 53. After 2 hours was 5 ml of hydrogen gas generated (equivalent to 97 ml/g Si). From example 26 was 56% of losartan released from the particles, comparing to free losartan powder, during 2 hours. After 3 days was 28 ml hydrogen gas generated (equivalent to 546 ml/g Si).

### Example 65 Tablets Comprising Amorphous cCVD-SP Comprising 5% Rapamycin

Each tablet comprises:
Amorphous cCVD-SP comprising rapamycin (5% weight load) (from Example 49, 100 mg) Microcrystalline cellulose 360 mg
Crosscaramellose (Na) (AcDiSoI) 20 mg
Stearic acid 20 mg

All ingredients are blended. A tablet is compressed, Tablet diameter 10 mm Tablet weight: 500 mg. Rapamycin content: 5 mg.

### Example 66 Injection Suspension Comprising Amorphous cCVD-SP Comprising 5% Rapamycin

Amorphous cCVD-SP comprising rapamycin (5% weight load) are prepared from sterile cCVD-SP and sterile rapamycin analogous to the procedure in example 49 using an aseptic production process.

The sterile particles (100 mg) are suspended in a sterile solution of isotonic glucose solution (50 ml, 5% w/v) by sonication for 10 minutes under aseptic conditions. The suspensions are aseptically filled into injection vials (5 ml). Each vial contains 10 mg particles.

### Example 67 Chemical-Physical Stability of Aggregated Amorphous cCVD-SP

cCVD-SP of amorphous form (batch no. H18) were suspended in different solutions at a concentration of 1-5 mg/ml. Samples were withdrawn after 5 hours and 4 days to assess the stability of aggregated particles in solution by size measurements. Single particles have a size of 20-50 nm as seen from SEM images, while the aggregated particles made up of the smaller single particles have a size around 200 nm as measured with DLS.

Chemical stability was tested in PBS buffer of pH 7.4, and physical stability was tested by shaking the sample vial by hand, with ultrasound bath treatment for up to 15 minutes, exposure to elevated temperature (37° C.) in a water bath and vigorous magnetic stirring.

The agglomerated particles in purified water gave a hydrodynamic size of 282 nm (PDI: 0.287) after shaking the vial and a size of 209 nm (PDI: 0.189) after ultrasound treatment. Thus, ultrasound treatment readily disperse weakly bonded large agglomerates but do not separate the particle aggregate into single particles. After 5 hours and 4 days immersion of amorphous aggregated cCVD-SP in PBS at room temperature, the hydrodynamic size (after 1 minute ultrasonication treatment) was 264 nm (PDI: 0.352) and 323 nm (PDI: 0.479), respectively. In purified water and PBS at 37ºC, the hydrodynamic size after 5 hours (and 1 minute ultrasonication) was 249 nm (PDI: 0.238) and 344 nm (PDI: 0.463), respectively. Immersion in PBS at 37° C. with vigorous magnetic stirring (followed by 1 minute ultrasonication) resulted in hydrodynamic size of 446 nm (PDI: 0.492) after 5 hours and 469 nm (PDI: 0.522) after 4 days. PBS, elevated temperature treatment and magnetic stirring do not make the stable aggregates fall apart but increase the formation of large particles made up of weakly bonded agglomerates.

### Example 68 Stability of Aggregated Amorphous cCVD-SP in the Presence of Surfactants and Albumin

The experiments were performed as in example 67. Amorphous aggregate particles (batch no. H18) were immersed in purified water and PBS with addition of 0.1% (w/v) Pluronic F-127 (Sigma) or Polysorbate 80 (Apotekproduksjon), or 4% (w/v) albumin from human serum (≥96%, Sigma). All samples were treated for 1 min in ultrasound bath before measurement of hydrodynamic size.

The particles immersed in water with addition of albumin, Pluronic F-127 and Polysorbate 80 gave hydrodynamic sizes of 264 nm (PDI: 0.215), 221 nm (PDI: 0.168), 193 nm (PDI: 0.115) after 5 hours and 277 (PDI: 0.243), 292 nm (PDI: 0.269), 234 nm (PDI: 0.226) after 4 days. The particles immersed in PBS with addition of albumin, Pluronic F-127 and Polysorbate 80 gave hydrodynamic sizes of 266 nm (PDI: 0.192), 186 nm (PDI: 0.145), 185 nm (PDI: 0.122) after 5 hours and 291 (PDI: 0.244), 330 nm (PDI: 0.383), 193 nm (PDI: 0.139) after 4 days.

Aggregated particles are stable in terms of not collapsing into single particles. Agglomeration in PBS is not seen as extensively after addition of Pluronic F-127, Polysorbate 80 or Albumin as without these additions. These substances are likely to form adsorption coatings that stabilize the particles in PBS solutions.

### Example 69 Stability of Aggregated Amorphous cCVD-SP in an Artificial In Vitro Model of Blood

The experiment was performed as in example 67. Amorphous aggregate particles (batch no. H18) were immersed in an in vitro blood model containing PBS with 4% (w/v) albumin from human serum (≥96%, Sigma) kept in water bath at 37° C.

Hydrodynamic size measured after 5 hours, following shaking by hand, to 319 nm (PDI: 0.288) and, following 1 minute ultrasonication, to 310 nm (PDI: 0.231). Some agglomeration of the particles is seen in artificial blood, as compared to pure water.

1. A process for preparing silicon particles comprising at least one drug substance, wherein said process comprises the steps:
a) preparing silicon particles via chemical vapor deposition (CVD);
b) loading the silicon particles prepared in step a) with at least one drug substance.

2. A process as claimed in claim 1, wherein said silicon particles are centrifuge Chemical Vapor Deposition Silicon Particles (cCVD-SP) and said CVD in step a) is performed in a reactor comprising a reactor body and a rotation device operatively arranged to the reactor, wherein the rotation device is configured to rotate the reactor around an axis during production.

3. A process as claimed in claim 1 or 2, wherein step a) does not comprise milling the particles.

4. A process as claimed in any of claims 1 to 3, wherein said particles are non-etched particles.

5. A process as claimed in any of claims 1 to 4, wherein step b) comprises mixing the silicon particles prepared in step a) with at least one drug substance.

6. A process as claimed in any of claims 1 to 5, where said silicon particles comprise at least 50 wt % crystalline silicon, relative to the total weight of silicon.

7. A process as claimed in any of claims 1 to 6, wherein said silicon particles comprise at least 50 wt % amorphous silicon, relative to the total weight of silicon.

8. A process as claimed in any of the claims 1 to 7, wherein said silicon particles comprise at least 50 wt % elemental silicon, relative to the total weight of silicon.

9. A process as claimed in any of claims 1 to 8, wherein said at least one drug substance is in the form of a cyclodextrin complex.

10. A process as claimed in any of the claims 1 to 9 where said at least one drug substance is selected from the group consisting of anticancer drugs, drugs with effect on the immune system, antifungal drugs, antibiotics, antiviral drugs, drugs for treatment of CNS related diseases, antidiabetic drugs, drugs for treatment of pain and steroid-based drugs.

11. A process as claimed in any of the claims 1 to 9 where said at least one drug substance is selected from the group consisting of atorvastatin, simvastatin, losartan, valsartan, candesartan, enalapril, atenolol, propranolol, hydrochlotiazide, cyclosporine, amphotericin B, dilthiazem, phenoxymethylpenicillin, azithromycin, rapamycin, griseofulvin, chloramphenicol, erythromycin, acyclovir, nystatin, phenytoin, phenobarbital, ampicillin, celecoxib, prednisolon and metformin.

12. Silicon particles comprising at least one drug substance prepared according to the process of any of claims 1 to 11.

13. A pharmaceutical composition comprising silicon particles as defined in claim 12 and one or more pharmaceutically acceptable carriers, diluents or excipients.

14. A pharmaceutical composition as claimed in claim 13, wherein the composition is formulated for oral administration

15. A pharmaceutical composition as claimed in claim 14, wherein the composition is in the form of a tablet, capsule or suspension.

16. A pharmaceutical composition as claimed in claim 13, wherein the composition is formulated for subcutaneous administration.

17. A pharmaceutical composition as claimed in claim 13, wherein the composition is formulated for parenteral administration.

18. Silicon particles as claimed in claim 12 or a pharmaceutical composition as claimed in any of claims 13 to 17 for use in therapy.

19. Silicon particles as claimed in claim 12 or a pharmaceutical composition as claimed in any of claims 13 to 17 for use in drug delivery.
